(19) United States
(12) Patent Application Publication
(10) Pub. No.: US 2016/0038516 A1
(43) Pub. Date: Feb. 11, 2016

(54) COMBINATION CANCER THERAPY USING BISPHOSPHONATES AND ANTI-EGFR AGENTS

(71) Applicants: Goutham Narla, New York, NY (US); Jameel Iqbal, (US); Mone Zaidi Consulting, Inc., RIVERDALE, NY (US)

(72) Inventors: Mone Zaidi, Riverdale, NY (US); Goutham Narla, New Rochelle, NY (US); Jameel Iqbal, New York, NY (US)

(21) Appl. No.: 14/409,136

(22) PCT Filed: Jun. 25, 2013
(86) PCT No.: PCT/US2013/047666

§ 371 Date: Dec. 18, 2014

Related U.S. Application Data
(60) Provisional application No. 61/789,733, filed on Mar. 15, 2013.
(60) Provisional application No. 61/664,105, filed on Jun. 25, 2012.
(60) Provisional application No. 61/664,105, filed on Jun. 25, 2012.

(51) Int. Cl. A61K 31/675; A61K 31/538; A61K 45/06; A61K 31/517

(52) U.S. Cl. CPC A61K 31/675; A61K 31/517; A61K 31/538; A61K 45/06

(57) ABSTRACT

The present invention relates to combination therapies for the treatment of EGFR-related diseases, particularly EGFR-related cancers. This invention also relates to a method of enhancing the efficacy of an EGFR family member antagonist and therapeutic methods for subjects who are refractory to treatment with an EGFR family member antagonist. The invention also relates to pharmaceutical compositions useful for treatment of EGFR-related diseases.

This application claims priority from United States provisional applications 61/664,105 and 61/789,733, filed on Jun. 25, 2012 and Mar. 15, 2013, respectively. The disclosures of the priority applications are incorporated herein by reference in their entirety.

### BACKGROUND OF THE INVENTION

Cancer is one of the leading causes of death in the United States. Yet, a significant proportion of cancer patients remain with no curative treatment as traditional cytotoxic therapy has often limited efficacy. Uncovering molecular pathways driving the development and progression of cancer, with further validation of therapeutic approaches in appropriate genetic models can potentially establish targeted treatment to appropriately selected patients. An example of such an approach in targeted molecular therapy is directed against epidermal growth factor receptor signaling (EGFR) which has become a mainstay for the treatment of chemoresistant metastatic lung, pancreatic and colorectal adenocarcinoma. The clinical utility of anti-EGFR based strategies is, however, ultimately limited by universal development of either primary or acquired drug resistance and by the fact that these agents at best result in partial responses and prolong survival by months.

### SUMMARY OF THE INVENTION

This invention is based on inventors' discovery that bisphosphonates can enhance the efficacy and response to therapies targeting an epidermal growth factor receptor (EGFR) family member in cancer patients. A first aspect of the present invention provides a method of treating cancer, comprising identifying a cancer subject who has a mutation in an EGFR family member, and administering to the subject an EGFR family member antagonist and a bisphosphonate that can bind to the EGFR family member.

Another aspect of the present invention provides a method of enhancing the efficacy of an EGFR family member antagonist in a subject, comprising administering to the subject the EGFR family member antagonist and a bisphosphonate that can bind to an EGFR family member.

A further aspect of the present invention provides a method of treating cancer in a subject, wherein the cancer is characterized by a T790M mutation in EGFR or a T798M or T798I mutation in HER2, comprising administering to the subject a bisphosphonate that binds to an EGFR family member.

Yet another aspect of the present invention provides a pharmaceutical composition comprising an EGFR family member antagonist and a bisphosphonate. The EGFR family member antagonist and bisphosphonate may be present in amounts such that the EGFR family member antagonist and the bisphosphonate synergize to induce either cell cycle arrest or apoptosis. Also provided are uses of the compositions for the manufacture of medicament for use in the treatment methods of this invention.

The methods and pharmaceutical composition may be used for treating a subject with cancer. In some embodiments of these methods and compositions, the subject has a mutation in EGFR/ErbB-1/HER1, and may have, for example, lung cancer (e.g., non-small cell lung cancer), breast cancer, or colorectal cancer. In other embodiments, the subject has a mutation in HER2, and may have, for example, breast cancer, ovarian cancer, stomach cancer, or uterine cancer (e.g., endometrial cancer). In further embodiments, the subject has a mutation in Her3, and may have, for example, breast cancer, prostate cancer, or bladder cancer.

As used herein, the term “treating cancer” refers to treatment of an existing cancer, for example, by inhibiting or reducing cancer progression, metastasis, tumor growth; and to prevention of cancer, for example, by preventing or reducing the risk of carcinogenesis and cancer recurrence. A cancer patient refers to a patient who has been diagnosed of having cancer, or to a patient who is at risk of having cancer, for example, due to genetic predisposition, including having the genetic mutations mentioned herein. Thus, the treatment methods of this invention encompass treatment of existing cancer using the pharmaceutical compositions of this invention, as well as the prophylactic use of the pharmaceutical compositions in patients who are susceptible to developing cancer. Cancer that can be treated by the methods and compositions of this invention include, for example, lung cancer (e.g., non-small cell lung cancer), breast cancer, or colorectal cancer, ovarian cancer, stomach cancer, and uterine cancer (e.g., endometrial cancer).

In some embodiments of the present methods and compositions, the anti-cancer therapeutics (an anti-EGFR family member antagonist and a bisphosphonate) may be administered before the cancer metastasizes, e.g., before the cancer metastasizes to a bone, in the subject. In some embodiments, the subject may be refractory to a prior treatment with an EGFR family member antagonist, such as erlotinib, lapatinib, trastuzumab or pertuzumab. Such a subject may have acquired one or more of genetic mutations such as a T790M mutation in EGFR; a G309A, G309E, S310F, L755S, T798M, or T798I mutation; or HER2 or MET amplification. The methods and compositions of this invention are especially effective in treating such patients.

In some embodiments of the present methods and compositions, the subject (i.e., patient) may harbor a mutation in an EGFR family member (e.g., EGFR/ EGFR/ErbB-1/HER1; HER2; or HER3) in the catalytic domain. In some embodiments, the EGFR family member may be an EGFR and may be overexpressed, and/or have one or more of the following mutations: L858R, a deletion in exon 19 (such as deletion of residues 746-750), insertions in exon 20 (see, e.g., Arcila et. al., Mol. Cancer Ther. (2013) 12(2): 220-9, incorporated herein by reference), and T790M. Alternatively, the EGFR family member may be a HER2 and may be expressed, and/or have a T798M, T798I, G309A, G309E, S310F, R678Q, L755S, L755W, I767M, D769H, D769Y, V777L, P780ins, V835F, V842I, R896C, or G1201V point mutation, or an in-frame deletion (such as deletion of residues 755-759).

In some embodiments of the present methods and compositions, the bisphosphonate that can bind to the EGFR family member comprises one or more nitrogen atoms. For example, the bisphosphonate may comprise an imidazole ring. By way of example, the bisphosphonate may be zoledronate, minodronate, alendronate, ibandronate, risedronate, and incadronate, or an acid or any pharmaceutically acceptable derivative of any of the foregoing.

In some embodiments of the present methods and compositions, the EGFR family member antagonist binds to the catalytic domain of the EGFR family member. In some embodiments, the EGFR family member antagonist is erlotinib, gefitinib, lapatinib, neratinib or afatinib. Alternatively, the EGFR family member antagonist may bind to an extracellular domain of the EGFR family member. In such embodiments, the EGFR family member antagonist may, for example, be cetuximab, panitumumab, matuzumab, nimotuzumab, trastuzumab, pertuzumab, or NEUVAX (nelipepimut-S).

### BRIEF DESCRIPTION OF THE DRAWINGS

FIG. 1 provides the gene signature developed following affymetrix analysis of gene expression in response to bisphosphonate stimulation.

FIG. 2 provides the results of bisphosphonate-drug pairs by connectivity mapping. Bisphosphonates paired with several EGFR inhibitors.

FIG. 3 demonstrates that KEGG pathway analysis of the bisphosphonate gene signature establishes that bisphosphonates affect the EGFR signaling pathway.

FIG. 4 provides the results of an MTT assay that demonstrates how zoledronic acid (a bisphosphonate) decreases viability of lung cancer cell lines. Zoledronic acid effectively decreased viability of cells with either wild-type EGFR or mutant EGFR, including T790M EGFR.

FIG. 5 demonstrates that zoledronic acid displayed minimal or no effects on cell viability or apoptosis in the EGFR-negative lung cancer cell line H520 or the EGFR-low breast cancer cell line MCF-7 when exposed for up to 72 hours (FIG. 5A). However, cell survival was significantly attenuated in Caco-2 colon cancer and H1666 lung cancer cells that express the wild type EGFR at moderate and high levels, respectively (FIG. 5B). In contrast, a lung cancer cell line A549 that expresses wild type EGFR, but is driven by a Gl2S ras mutation was poorly responsive to both zoledronic acid and erlotinib (FIG. 5C).

FIG. 6 evaluates the effectiveness of various bisphosphonate compounds in decreasing viability of lung cancer cells comprising L858R mutant EGFR. Each bisphosphonate compound that effectively decreased the viability of lung cancer cells comprising L858R mutant EGFR comprised at least one nitrogen atom (“N-BP”).

FIG. 7 evaluates the effectiveness of various bisphosphonate compounds in decreasing viability of lung cancer cells comprising EGFR with an Exon 19 deletion (del 746-750). Each bisphosphonate compound that effectively decreased the viability of lung cancer cells comprising EGFR with an Exon 19 deletion (del 746-750) comprised at least one nitrogen atom (“N-BP”).

FIG. 8 demonstrates that minodronic acid and zoledronic acid, which each comprise nitrogen-containing rings, are effective at decreasing the viability of lung cancer cells containing either L858R mutant EGFR or EGFR with an Exon 19 deletion (del 746-750).

FIG. 9 demonstrates the cellular mechanisms of action of zoledronic acid on lung cancer cells. Zoledronic acid increased apoptosis of lung cancer cells comprising L858R EGFR. Zoledronic acid arrested growth of lung cancer cells comprising EGFR with an Exon 19 deletion (del 746-750). Zoledronic acid did not have an effect on lung cancer cells with a Ras mutation.

FIG. 10 demonstrates that bisphosphonates inhibit growth by causing cell cycle arrest and a modest stimulation of apoptosis. PARP: poly ADP ribose polymerase; p-AKT: phosphorylated AKT; t-AKT: total AKT; t-ERK: total ERK; p-ERK: phosphorylated ERK; GAPDH: glyceraldehyde 3-phosphate dehydrogenase; PCNA: proliferating cell nuclear antigen.

FIG. 11 demonstrates that both alendronate (ALN) and risedronate (RIS) inhibited EGFR kinase activity in a cell-free system that used recombinant EGFR.

FIG. 12 describes the phosphorylation status of Y845, Y992, Y1045, Y1068, and Y1173 in response to zoledronic acid (ZA) and epidermal growth factor (EGF) administration. Statistics: Student's t-test; p values comparing treatments with ATP (A) or without ZA (B); *p<0.05; **p<0.01.

FIG. 13 depicts a cell-free in vitro kinase assay using the recombinant EGFR^{L858R}. Ris, ZA or Ert (doses as shown) were incubated for 30 minutes with receptor and EGFR kinase activity was measured. 50 ng recombinant EGFR^{L858R} protein (GENWAY) was incubated with zoledronic acid or risedronate at 4° C. for 30 minutes. Following addition of assay buffer (SIGMA) which contained the poly(Glu, Tyr) (4:1) substrate and ATP, the kinase reaction was allowed to proceed for 30 minutes at 30° C. An equal volume of Kinase-Glo substrate (PROMEGA) was then added, and luminescence read manually (TD-20/20, TURNER DESIGN).

FIG. 14 demonstrates that zoledronic acid and minodronic acid potently reduce the viability of EGFR mutation-positive cancer cells. In contrast, tiludronate does not affect either cell line.

FIG. 15 demonstrates that the inhibition by zoledronic acid on HCC827 cell viability was mirrored by an inhibition of colony formation

FIG. 16 demonstrates that the T790M mutation does not abolish the effect of bisphosphonate on decreasing the viability of lung cancer cells comprising L858R EGFR. PARP: poly ADP ribose polymerase; P-AKT: phosphorylated AKT; T-AKT: total AKT; T-ERK: total ERK; P-ERK: phosphorylated ERK; pEGFR: phosphorylated EGFR; GAPDH: glyceraldehyde 3-phosphate dehydrogenase.

FIG. 17 demonstrates that erlotinib and zoledronic acid synergize to inhibit cell viability of lung cancer cells containing either L858R mutant EGFR or EGFR with an Exon 19 deletion (del 746-750).

FIG. 18 demonstrates that the effect of erlotinib was significantly enhanced when the drug was combined with minodronic acid, an imidazole-containing bisphosphonates (panel A). However, when combined with tiludronate, which does not have an imidazole ring, the synergistic inhibition was not observed (panel B).

FIG. 19 demonstrates that erlotinib and zoledronic acid synergize to enhance apoptosis of lung cancer cells containing EGFR with an Exon 19 deletion (del 746-750).

FIG. 20 provides Western analyses, which suggest that erlotinib and zoledronic acid synergize to enhance apoptosis of lung cancer cells containing either L858R mutant EGFR or EGFR with an Exon 19 deletion (del 746-750).

FIG. 21 demonstrates that erlotinib and zoledronic acid synergize to suppress colony formation of lung cancer cells.

FIG. 22 demonstrates that combinations of two bisphosphonate compounds (either zoledronic acid and risedronate or zoledronic acid and incadronate) are not synergistic.

FIG. 23 provides colony formation and cell viability assays in a L858R/T790M-derived lung adenocarcinoma cell line, H1975. Zoledronic acid inhibited colony formation, but erlotinib and tiludronate did not (panel A).

FIG. 24 demonstrates that zoledronic acid inhibited cell viability of the L858R/T790M double-mutant (H1975) in a concentration-dependent manner, but erlotinib did not (panel A). Erlotinib was able to inhibit cell viability and synergize with zoledronic acid in the cell line H3255 (L858R), which does not harbor the gatekeeper mutation T790M (panel B).

FIG. 25 demonstrates that the inhibitory effects of zoledronic acid on the L858R/T790M double-mutant (H1975) were exerted through the stimulation of apoptosis (panel A), as evident from sharp increases in PARP expression and reduced Akt phosphorylation (panel B). In contrast, there were minimal changes in the expression of cell cycle proteins in the double mutant cells (panel B). PARP: poly ADP ribose polymerase; P-AKT: phosphorylated AKT; T-ERK: total ERK; P-ERK: phosphorylated ERK; GAPDH:

glyceraldehyde 3-phosphate dehydrogenase; PCNA: proliferating cell nuclear antigen.

FIG. 26 examines the effects of zoledronic acid and erlotinib on tumor growth in vivo. Erlotinib and zoledronic acid each, individually, slowed tumor growth over the first six or five treatment periods, respectively, before causing a reduction in tumor size (panel A). The combination of erlotinib and zoledronic acid, however, showed relatively little tumor growth following the first treatment period and caused a dramatic reduction in tumor size beginning in the second treatment period. Both Western analysis (panel B) and immunohistochemistry (panel C) demonstrated that erlotinib and zoledronic acid each inhibited AKT phosphorylation, and the combination of erlotinib and zoledronic acid synergized to inhibit AKT phosphorylation. Erlotinib alone did not inhibit ERK phosphorylation, while zoledronic acid was able to inhibit ERK phosphorylation. The combination of erlotinib and zoledronic acid, however, synergistically inhibited ERK phosphorylation. p-AKT: phosphorylated AKT; AKT: total AKT; ERK: total ERK; p-ERK 1/2: phosphorylated ERK 1/2.

FIG. 27 demonstrates the effect of erlotinib (Ert), zoledronic acid (ZA), or Ert+ZA on tumor growth in BALB/c nu/nu mice transplanted with HCC827 cells, shown as a change in tumor volume in individual mice (Waterfall plot) (A) or as ratio of tumor volume at day zero in groups of mice (B). Whereas Ert and ZA prevent tumor growth, the two drugs in combination cause tumor regression. (C) Apoptotic cells determined by TUNEL staining were counted for each treatment regimen. (e) The number of blood vessels in each tumor was determined by immunolabeling for CD31 in the same sections from the four groups of mice, with number of blood vessels quantitated per 40× field. Statistics: Student's t-test with Bonferroni's correction. Each treatment compared with DMSO at day 9 (̂p<0.05, ̂̂p<0.01) and ZA+Ert versus Ert and/or ZA alone (*p<0.05, **p<0.01). n=8 mice per group.

FIG. 28 depicts the effect of bisphosphonates on HER2 signaling. (A) Erlotinib (Ert) zoledronic acid (ZA), risedronate (Ris), minodronic acid (MA), tiludronate (Til) inhibit the tyrosine kinase activity of Her2 in a cell-free in vitro kinase assay using the recombinant Her2. EGFR^{L858R} (Ert L858R) was included as a control. (B) Western analysis demonstrates that the MDA-231 breast cancer cell line overexpresses EGFR, and the BT474 breast cancer cell line overexpressed HER2. (C) ZA decreases the viability of EGFR-expressing breast cancer cells (MDA-231). (D) ZA decreases the viability of HER2-expressing breast cancer cells (BT474). The non-N-containing bisphosphonate tiludronate (Til) has no effect on either cell line, demonstrating the specificity of the observed effect.

FIG. 29 depicts Western blots showing the effect of ZA on unprenylated Rap-1α (u-Rap-1α) accumulation in A549, H1975, HCC827 and H3255 cells, as evidence of FPPS inhibition (A). Cell viability inhibition and u-Rap-la accumulation (B) in response to bisphosphonates do not correlate (B).

FIG. 30 demonstrates that bisphosphonates induce apoptosis in EGFR and HER2 expressing breast cancer cells. Flow cytometry showing cell cycle profile of the EGFR-expressing MDA-231 (panel A), the HER2 expressing BT-474 (panel B) and the EGFR/HER2 negative MDA-453 (panel C) breast cancer cell lines. As shown by the increased sub G1 fraction, Zoledronic acid induces apoptosis only in the EGFR and HER expressing cell lines. The non-N-containing bisphosphonate tiludronate (Til) has no effect in any of the breast cell lines tested.

FIG. 31 demonstrates that bisphosphonates decrease colony formation in EGFR expressing colon cancer cells. Clonogenic assays in the EGFR negative (SW620, panel A) and EGFR expressing (SW480, panel B) colon cancer cell lines were performed in the presence of increasing concentrations of Zoledronic Acid (ZA). While Zoledronic acid reduces colony formation in EGFR-expressing cells (SW480), it has no effect in EGFR negative (SW620) colon cancer cell line. The non-N-containing bisphosphonate tiludronate (Til) has no effect on any of the colon cancer cell lines tested.

### DETAILED DESCRIPTION OF THE INVENTION

Bisphosphonates have been used to prevent bone loss in a subject such as patients suffering from osteoporosis. Bisphosphonates are often co-administered with other pharmaceutical therapies to treat diseases affecting the bone or when the other pharmaceutical therapy places the subject at risk for bone loss. We have discovered, however, that certain bisphosphonates can be effective in tissues other than bone. We have discovered that these bisphosphonates bind to the intracellular region of a member of the epidermal growth factor receptor (EGFR) family (e.g., EGFR/ErbB-1/Her1, and Her2/Neu/ErbB-2) and inhibit its signaling pathway. Such bisphosphonates may inhibit cellular growth and promote apoptosis by inducing cell cycle arrest.

We have also discovered that the bisphosphonates of this invention can bind particularly well to clinically relevant EGFR and Her2 mutants, and can be structurally accommodated in the ATP binding pocket of the receptor together with an EGFR family member-targeting tyrosine kinase inhibitor (TKI). And we have found that the bisphosphonates can consequently synergize with the TKI in inhibiting EGFR function and downregulating the AKT and ERK signaling pathways.

Accordingly, the bisphosphonates of this invention can be used alone, or with another therapeutic agent (e.g., another EGFR family member antagonist), in treating diseases mediated by a mutated or overexpressed EGFR family member, such as cancers (e.g., hematopoietic cancer, including lymphoma and leukemia; tumors, including carcinomas and sarcomas; mesothelioma, squamous cell neoplasms, gliomas). The bisphosphonates of this invention are especially useful as therapeutic agents in treating cancer patients who are refractory to another EGFR family targeted therapy. In addition, bisphosphonates can be used in conjunction with anti-EGFR and anti-Her2 based therapies as first line treatment.

### EGFR Family Members and Cancers

The EGFR family is a family of tyrosine kinase cell surface receptors that include EGFR, HER2, HER3, and HER4. The receptors bind to growth factors such as epidermal growth factor (EGF), EGF-like proteins, and neuregulins and transduce signals that stimulate cell division. It has been well documented that mutated or overactive EGFR family members are related to cancer development including tumorigenesis, tumor maintenance, tumor metastasis, and recurrence.

We have now discovered that the bisphosphonates of this invention bind to the ATP-binding pocket in the intracellular domain of an EGFR family member such as EGFR and HER2 and interrupt their signal transduction. Thus, the present bisphosphonates can be used to inhibit EGFR family functions and to treat cancers that are driven, maintained or promoted by an abnormal EGFR family member, such as a mutant, overactive or overexpressed EGFR, HER2, HER3 or HER4.

EGFR family member mutations or dysregulation have been linked to a number of epithelial cancers. For example, EGFR overactivity caused by a L858R point mutation, a T790M point mutation, an exon 20 insertion (see, e.g., Arcila et. al., Mol. Cancer Ther. (2013) 12(2): 220-9, incorporated herein by reference) or an exon 19 deletion in the catalytic domain has been associated with lung cancer (e.g., nonsmall cell lung cancer), colorectal cancer, and glioblastoma. HER2 dysregulation caused by mutations such as a T798M point mutation have been associated with breast cancer, stomach cancer, uterine cancer, ovarian cancer. Additional mutations in HER2 include T798I, G309A, G309E, S310F, R678Q, L755S, L755W, I767M, D769H, D769Y, V777L, P780ins, V835F, V842I, R896C, and G1201V point mutations, or an in-frame deletion (such as deletion of residues 755-759). We have discovered that the present bisphosphonates can bind to EGFR family members including their mutants, such as those described above. Thus, the present bisphosphonates are particularly useful therapeutic agents in treating EGFR family member-mediated cancers in a cancer patient (e.g., a human, a farm animal, or a household pet).

In some embodiments, the EGFR family member targeted by the compounds and methods of this invention is wild-type. A wild-type gene or gene product refers to a gene or gene product that is most frequently observed in a population and is thus arbitrarily designed as the “normal” or “wild-type” form of the gene or gene product. Wild-type sequences for human EGFR and human HER2 are shown in SEQ ID NO:1 and SEQ ID NO:2, available in GenBank under Accession Numbers NP_{—}005219 and NP_{—}004439, respectively.

In other embodiments, the EGFR family member targeted by the compounds and methods of this invention is a mutant such as those described herein. A mutant gene or gene product refers to a gene or gene product that displays modifications in sequence and/or functional properties when compared to the wild-type gene or gene product. EGFR family members isolated from many cancers have been shown to contain activating mutations, in which the mutant protein is active in the absence of an activating signal (e.g., constitutively active). The present bisphosphonates are efficacious in inhibiting the functions of these mutants and thereby in treating these cancers. For example, the present bisphosphonates can target a mutant human EGFR comprises an L858R point mutation, a T790M point mutation, an exon 20 insertion, and/or an exon 19 deletion in the catalytic domain (e.g., a deletion of amino acids 746-750). They can also target a mutant human HER2 comprising a T798M, T798I, G309A, G309E, S310F, R678Q, L755S, L755W, I767M, D769H, D769Y, V777L, P780ins, V835F, V842I, R896C, or G1201V point mutation or an in-frame deletion (such as deletion of residues 755-759).

Cancers that can be treated by the present bisphosphonates include those that are mediated by an EGFR family member. Such cancers include, without limitation, epithelial cancers such as lung cancer (e.g., nonsmall cell lung cancer), breast cancer, prostate cancer, ovarian cancer, uterine cancer, head and neck cancer, glioblastoma, stomach cancer, or colorectal cancer.

The present bisphosphonates are particularly useful in treating cancers that are refractory to EGFR family member antagonist treatment. The bisphosphonates may restore the cancer's sensitivity to the previously used antagonist. In some embodiments, the cancer is refractory to an EGFR antagonist (e.g., gefitinib, erlotinib, lapatinib, neratinib, afatinib, panitumumab, matuzumab, nimotuzumab, and cetuximab) treatment. In some embodiments, the cancer is refractory to an HER2 antagonist (e.g., trastuzumab, pertuzumab, NeuVax (Galena Biopharma), lapatinib, neratinib, afatinib and cetuximab). The bisphosphonates can be used alone, or together with another EGFR family member antagonist, including those to which the cancers have become refractory.

In some embodiments, it may be preferred to identify a cancer patient who has a mutation in an EGFR family member such as EGFR and HER2. EGFR and HER2 mutations can be readily identified by well known methods in the art, including those involving gene sequencing or the use of mutant-specific antibodies. See, e.g., Thomas et al., Nature Medicine 12(7):852-855 (2006). The EGFR family members may also be activated by overexpression of the receptor, overexpression of a ligand, and overexpression or mutation in a binding partner.

### The Bisphosphonates of the Invention

It has been shown for the first time that certain bisphosphonates bind specifically to EGFR family members such as EGFR and HER2, including their mutant forms.

Bisphosphonates have the following common core structure:

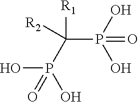

We have discovered that bisphosphonates containing one or more nitrogen atom can bind to EGFR family members. Without wishing to be bound by theory, the length of the nitrogen containing chain or the configuration (e.g., cyclic configuration) may affect the ability of the bisphosphonate to bind the EGFR family member. For example, the bisphosphonate may comprise a nitrogen-containing ring such as an imidazole ring (e.g., imidazo[1,2-a]pyridine). Examples of the present bisphosphonates include alendronate, ibandronate, risedronic acid, incadronate, minodronic acid, and zoledronic acid, and their respective acid or salt forms. Optionally, the bisphosphonate is one that does not have high affinity for bone (hydroxyapatite crystal) but has high affinity for EGFR. If the charge of the bisphosphonate is reduced, plasma concentration will increase and avidity to hydroxyapatite crystal will decrease. Thus, a bisphosphonate with a nitrogen-containing ring may be used to achieve such goals.

In a combination therapy of the invention, the bisphosphonate and an EGFR family member antagonist can be administered simultaneously in separate formulations or as a single pharmaceutical formulation. The bisphosphonate and the EGFR family member antagonist may also be administered sequentially, with the bisphosphonate being administered before or after the antagonist.

The EGFR family member antagonists and bisphosphonates, as used herein, include any pharmaceutically acceptable derivatives, such as pharmaceutically acceptable salts, esters, and salts of such esters, that have the same biological functions as the original compound.

Pharmaceutically acceptable salts of the compounds of this invention include, for example, those derived from inorganic and organic acids and bases. Examples of suitable acids include hydrochloric, hydrobromic, sulfuric, nitric, perchloric, fumaric, maleic, phosphoric, glycolic, lactic, salicylic, succinic, toluene-p-sulfonic, tartaric, acetic, citric, methanesulfonic, formic, benzoic, malonic, naphthalene-2-sulfonic and benzenesulfonic acids. Other acids, such as oxalic, while not in themselves pharmaceutically acceptable, may be employed in the preparation of salts useful as intermediates in obtaining the compounds of the invention and their pharmaceutically acceptable acid addition salts. Salts derived from appropriate bases include alkali metal (e.g., lithium, sodium, potassium, etc.), alkaline earth metal (e.g., beryllium, magnesium, calcium, etc.), ammonium, and N—(C₁₋₄ alkyl)₄⁺ salts.

This invention also envisions the “quaternization” of any basic nitrogen-containing groups of the compounds disclosed herein. The basic nitrogen can be quaternized with any agents known to those of ordinary skill in the art including, for example, lower alkyl halides, such as methyl, ethyl, propyl and butyl chloride, bromides and iodides; dialkyl sulfates including dimethyl, diethyl, dibutyl and diamyl sulfates; long chain halides such as decyl, lauryl, myristyl and stearyl chlorides, bromides and iodides; and aralkyl halides including benzyl and phenethyl bromides. Water or oil-soluble or dispersible products may be obtained by such quaternization.

Due to our discovery that the present bisphosphonates do not just act on bone to prevent bone loss, but they also bind to an EGFR family member, health care providers can now use the present bisphosphonates in a subject suffering from cancer before the cancer metastasizes and therefore can be used be as adjunctive therapy in EGFR family member driven cancers, or even before the cancer metastasizes at all.

### Pharmaceutical Compositions and Administration Thereof

The present invention provides pharmaceutical compositions comprising a bisphosphonate of the invention and a pharmaceutically acceptable carrier. In some embodiments, the pharmaceutical composition comprises a therapeutically effective amount of an EGFR family member antagonist and the bisphosphonate.

A pharmaceutical acceptable carrier includes any and all appropriate solvents, dispersion media, coatings, surfactants, antioxidants, preservatives (i.e., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, e.g., Remington's Pharmaceutical Sciences, 18th Ed. Mack Printing Company, 1990, pp. 1289-1329, incorporated herein by reference). Except insofar as any conventional carrier is incompatible with the active ingredient, its use in the therapeutic or pharmaceutical compositions is contemplated.

The bisphosphonates are administered to a patient in a “therapeutically effective amount.” By that we mean the dosage of the bisphosphonate is sufficient to prevent cancer development, including cancer formation, growth, metastasis, maintenance, and/or recurrence.

The bisphosphonates can be administered orally, topically, parenterally, by inhalation or spray or rectally in dosage unit formulations containing conventional non-toxic pharmaceutically acceptable carriers, adjuvants and vehicles, either alone with another cancer therapeutic (such as an EGFR family member antagonist). The term parenteral as used herein includes percutaneous, subcutaneous, intravascular (e.g., intravenous), intramuscular, or intrathecal injection or infusion techniques and the like. In addition, there is provided a pharmaceutical formulation comprising one or more active ingredients (e.g., a bisphosphonate of the invention, and an EGFR family member antagonist, or both) and a pharmaceutically acceptable carrier. One or more active ingredients of the invention can be present in association with one or more non-toxic pharmaceutically acceptable carriers and/or diluents and/or adjuvants, and if desired other active ingredients. The pharmaceutical compositions containing active ingredients of the invention can be in a form suitable for oral use, for example, as tablets, troches, lozenges, aqueous or oily suspensions, dispersible powders or granules, emulsion, hard or soft capsules, or syrups or elixirs. The pharmaceutical compositions of the invention can also be used in combination with any of a variety of treatment modalities, such as chemotherapy, surgery, radiation therapy, or small molecule regimens.

In some embodiments, the pharmaceutical compositions of the present invention are administered over several hours (e.g., about 1, 2, 3, 4, 5, 6, 7, or 8 hours) through infusion, until the active ingredient (bisphosphonate) in the patient's blood has reached the desired level.

Compositions intended for oral use can be prepared according to any method known to the art for the manufacture of pharmaceutical compositions and such compositions can contain one or more such sweetening agents, flavoring agents, coloring agents or preservative agents in order to provide pharmaceutically elegant and palatable preparations. Tablets contain the active ingredient in admixture with non-toxic pharmaceutically acceptable excipients that are suitable for the manufacture of tablets. These excipients can be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets can be uncoated or they can be coated by known techniques. In some cases such coatings can be prepared by known techniques to delay disintegration and absorption in the gastrointestinal tract and thereby provide a sustained action over a longer period. For example, a time delay material such as glyceryl monosterate or glyceryl distearate can be employed.

Formulations for oral use can also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin or olive oil.

Aqueous suspensions contain the active materials in admixture with excipients suitable for the manufacture of aqueous suspensions. Such excipients are suspending agents, for example sodium carboxymethylcellulose, methylcellulose, hydropropyl-methylcellulose, sodium alginate, polyvinylpyrrolidone, gum tragacanth and gum acacia; dispersing or wetting agents can be a naturally-occurring phosphatide, for example, lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions can also contain one or more preservatives, for example ethyl, or n-propyl p-hydroxybenzoate, one or more coloring agents, one or more flavoring agents, and one or more sweetening agents, such as sucrose or saccharin.

Oily suspensions can be formulated by suspending the active ingredients in a vegetable oil, for example arachis oil, olive oil, sesame oil or coconut oil, or in a mineral oil such as liquid paraffin. The oily suspensions can contain a thickening agent, for example beeswax, hard paraffin or cetyl alcohol. Sweetening agents and flavoring agents can be added to provide palatable oral preparations. These compositions can be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents or suspending agents are exemplified by those already mentioned above. Additional excipients, for example sweetening, flavoring and coloring agents, can also be present.

Pharmaceutical compositions of the invention can also be in the form of oil-in-water emulsions. The oily phase can be a vegetable oil or a mineral oil or mixtures of these. Suitable emulsifying agents can be naturally-occurring gums, for example gum acacia or gum tragacanth, naturally-occurring phosphatides, for example soy bean, lecithin, and esters or partial esters derived from fatty acids and hexitol, anhydrides, for example sorbitan monooleate, and condensation products of the said partial esters with ethylene oxide, for example polyoxyethylene sorbitan monooleate. The emulsions can also contain sweetening and flavoring agents.

Syrups and elixirs can be formulated with sweetening agents, for example glycerol, propylene glycol, sorbitol, glucose or sucrose. Such formulations can also contain a demulcent, a preservative and flavoring and coloring agents. The pharmaceutical compositions can be in the form of a sterile injectable aqueous or oleaginous suspension. This suspension can be formulated according to the known art using those suitable dispersing or wetting agents and suspending agents that have been mentioned above. The sterile injectable preparation can also be a sterile injectable solution or suspension in a non-toxic parentally acceptable diluent or solvent, for example as a solution in 1,3-butanediol. Among the acceptable vehicles and solvents that can be employed are water, Ringer's solution and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid find use in the preparation of injectables.

The pharmaceutical compositions of the invention can also be administered in the form of suppositories, e.g., for rectal administration of the drug. These compositions can be prepared by mixing the active ingredient with a suitable non-irritating excipient that is solid at ordinary temperatures but liquid at the rectal temperature and will therefore melt in the rectum to release the drug. Such materials include cocoa butter and polyethylene glycols.

Pharmaceutical compositions of the invention can be administered parenterally in a sterile medium. The drug, depending on the vehicle and concentration used, can either be suspended or dissolved in the vehicle. Advantageously, adjuvants such as local anesthetics, preservatives and buffering agents can be dissolved in the vehicle.

Dosage levels of the order of from about 0.01 mg to about 140 mg per kilogram of body weight per day are useful in the treatment of the disease conditions described herein (about 0.5 mg to about 7 g per patient or subject per day). The amount of active ingredient that can be combined with the carrier materials to produce a single dosage form varies depending upon the host treated and the particular mode of administration. Dosage unit forms generally contain between from about 1 mg to about 500 mg of an active ingredient.

It is understood that the specific dose level for any particular patient or subject depends upon a variety of factors including the activity of the specific compound employed, the age, body weight, general health, sex, diet, time of administration, route of administration, and rate of excretion, drug combination and the severity of the particular disease undergoing therapy.

For administration to non-human animals, the composition can also be added to the animal feed or drinking water. It can be convenient to formulate the animal feed and drinking water compositions so that the animal takes in a therapeutically appropriate quantity of the composition along with its diet. It can also be convenient to present the composition as a premix for addition to the feed or drinking water.

The pharmaceutical compositions of the present invention can also be administered to a subject in combination with other therapeutic compounds to increase the overall therapeutic effect. The use of multiple compounds to treat an indication can increase the beneficial effects while reducing the presence of side effects.

Unless defined otherwise, all technical and scientific terms used herein have the same meaning as those commonly understood by one of skill in the art to which this invention belongs. In case of conflict, the present specification, including definitions, will control. Although methods and materials similar or equivalent to those described herein can be used in the practice or testing of the present invention, suitable methods and materials are described below. The materials, methods and examples are illustrative only, and are not intended to be limiting. All publications, patents and other documents mentioned herein are incorporated by reference in their entirety. Throughout this specification, the word “comprise” or variations such as “comprises” or “comprising” will be understood to imply the inclusion of a stated integer or groups of integers but not the exclusion of any other integer or group of integers.

### Examples

In order that this invention be more fully understood, the following examples are set forth. These examples are for the purpose of illustration only and are not to be construed as limiting the scope of the invention in any way. In the following examples, EGFR refers to EGFR/ErbB-1/Her1 unless otherwise specified.

### Example 1

### Connectivity Mapping Identifies the EGFR as a Bisphosphonate Target

To discover new mechanisms through which bisphosphonates might act, we interrogated the Connectivity Map (CMAP) (worldwide website at www.broad.mit.edu/cmap) using a novel bisphosphonate signature. The CMAP is a compendium of genome-wide gene expression data that allows functional connections between drugs, genes and diseases to be established. We cultured human osteoclasts derived from peripheral blood mononuclear cells (PBMC) obtained from three separate donors and exposed the cultures to vehicle, RANK-L (30 ng/ml), or RANK-L plus either alendronate or risedronate (10⁻⁸ M of either), two of the most commonly used bisphosphonates. Microarrays were performed on mRNA isolated from these cultured cells. We found that the expression of 486 genes was elevated by both bisphosphonates, while the expression of 176 genes was decreased by both bisphosphonates.

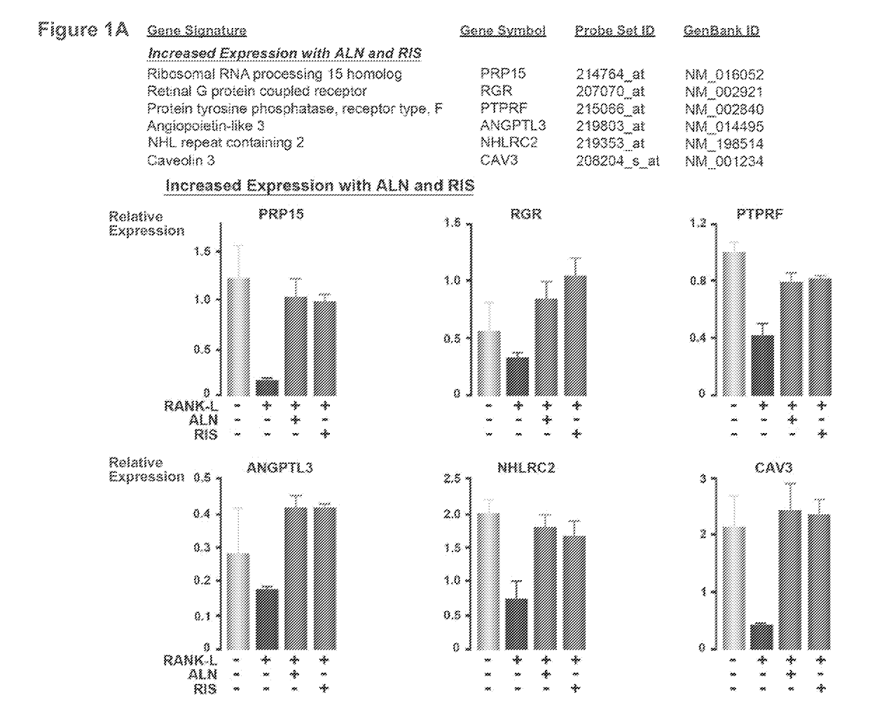

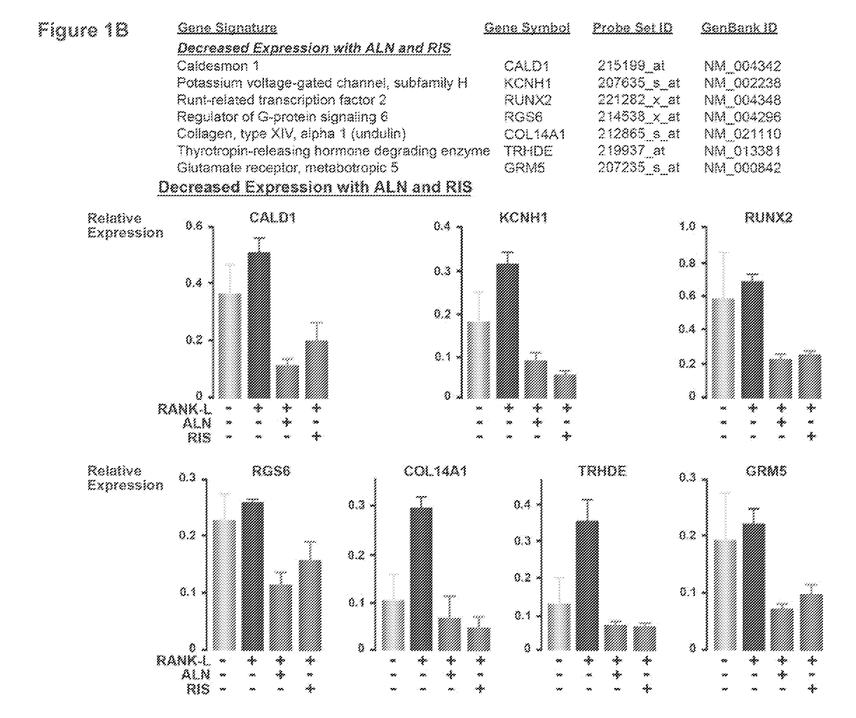

We then developed a bisphosphonate gene signature by identifying the genes that displayed the greatest statistical significance in being up- or down-regulated across both alendronate- and risedronate-treated samples (FIG. 1). Statistical comparisons were made utilizing the Student's t-test with Bonferroni's correction to yield p-values reflecting differences between RANK-L-induced gene expression and its alteration with both alendronate and risedronate. This approach identified genes that were affected in common by the two bisphosphonates, and allowed for the creation of a signature that was free from changes attributable solely to either agent.

The bisphosphonate signature consisted of six up-regulated genes (p<0.006), and seven down-regulated genes (p<0.0005) (FIG. 1). The microarray-derived gene signature was validated by qPCR (FIG. 1). Despite strong statistical associations with bisphosphonate exposure, none of the signature genes have established functions in osteoclasts. For example, while RUNX2 regulates osteoblast formation, there are no reports of its expression in osteoclasts.

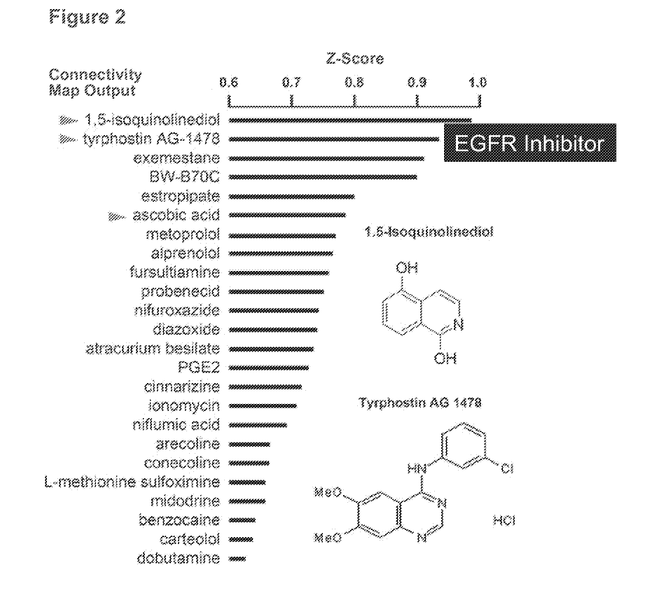

We interrogated CMAP with this gene signature to identify chemicals with similar genomic mechanisms to bisphosphonates. Drug-drug matching was performed by a nonparametric, rank-based algorithm using Kolmogorov-Smirnov statistic. Gene Set Enrichment Analysis (GSEA) examined shared mechanisms of action to reveal both mimics and anti-mimics of a given compound or disease. Of the chemicals displaying the highest enrichment scores, the top two were anti-cancer agents, namely the PARP inhibitor 1,5-isoquinolinediol, and a first-generation EGFR kinase inhibitor tyrphostin AG1478 (FIG. 2). These compounds were confirmed as “true” bisphosphonate mimetics using osteoclast formation assays in vitro. As with bisphosphonates, osteoclast formation was inhibited by both 1,5-isoquinolinediol and tyrphostin AG1478, the latter in a concentration-dependent manner (data not shown).

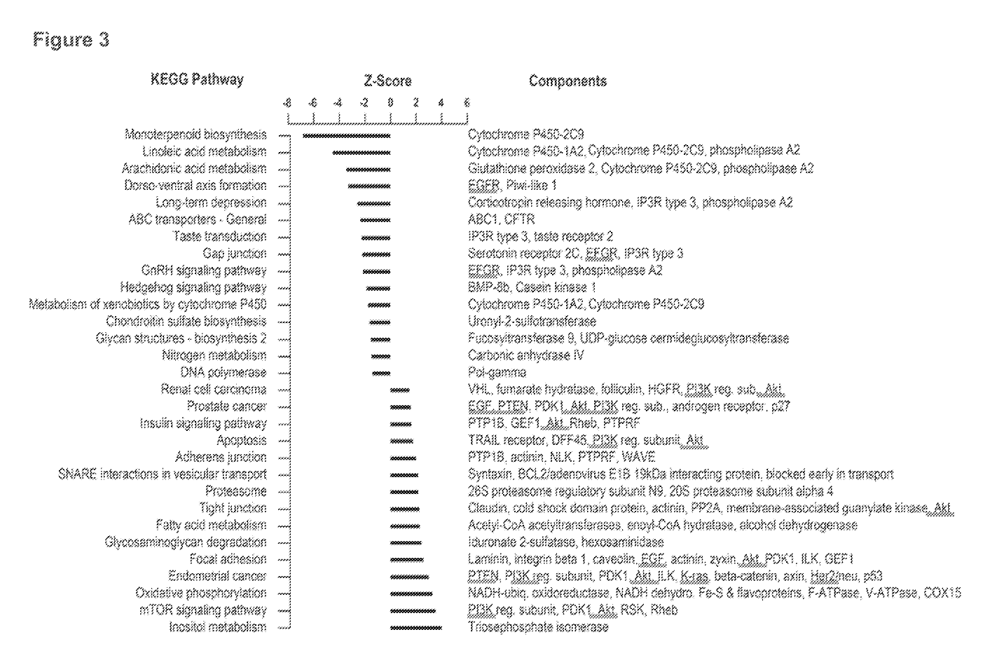

The identification of an EGFR inhibitor as a bisphosphonate mimic was intriguing because parallel analysis of the same microarray dataset using the KEGG Pathway Map demonstrated that 11 out of 30 pathways associated with bisphosphonate action contained EGFR signaling molecules, including the EGFR, EGF, PI3-kinase, Akt, PTEN and Rafl (FIG. 3). Together the data suggest that a hitherto unappreciated and common action of bisphosphonates might be to regulate EGFR signaling.

### Example 2

### Bisphosphonates Directly Inhibit the EGFR

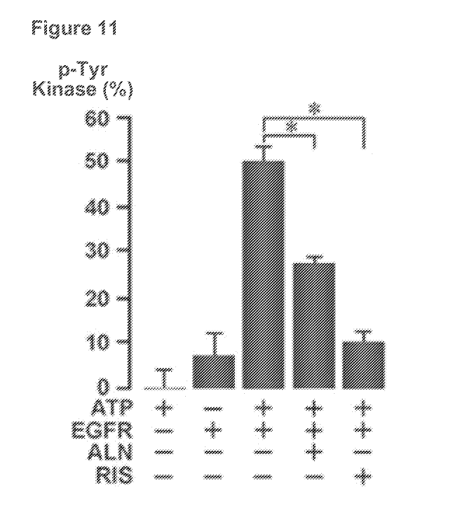

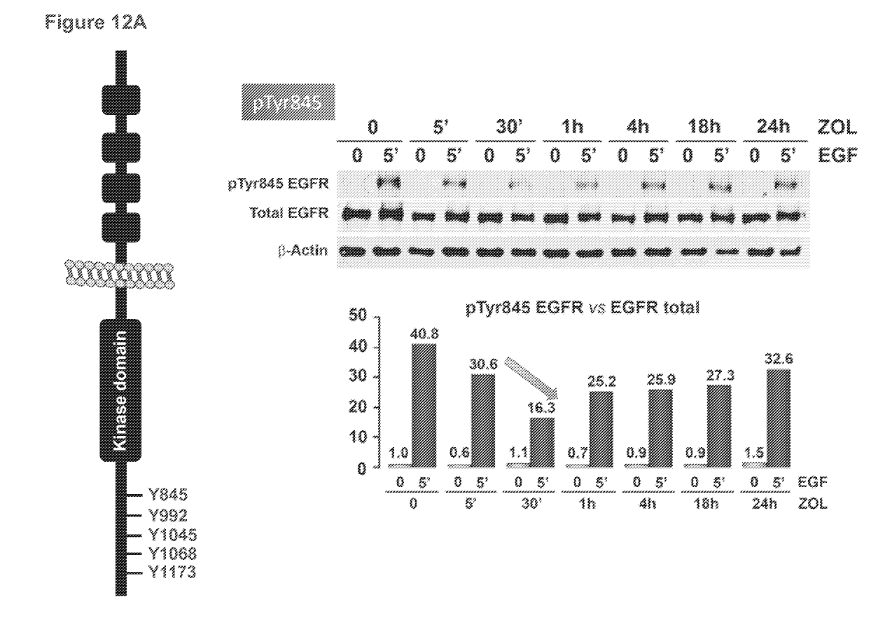

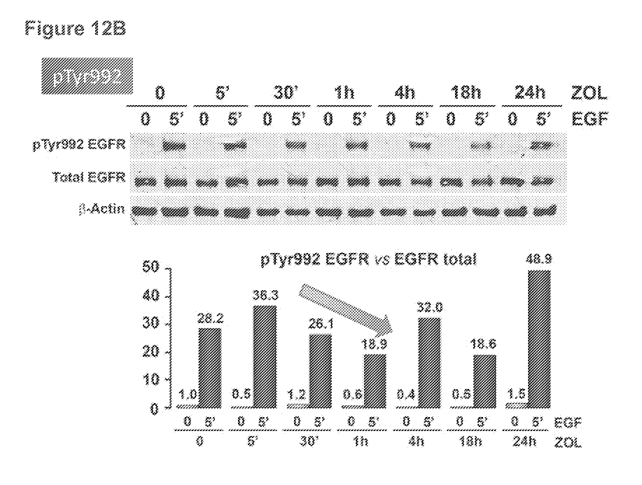

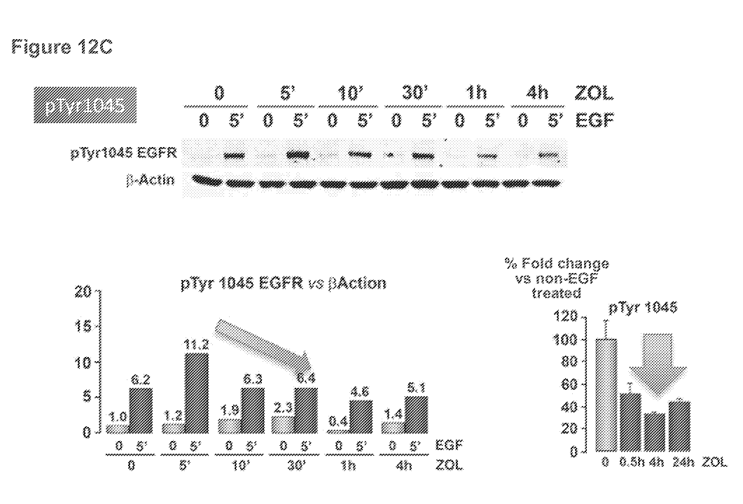

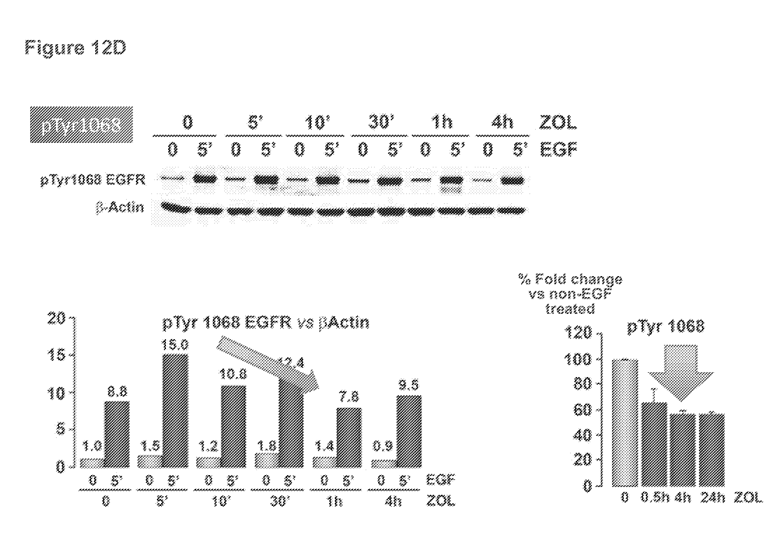

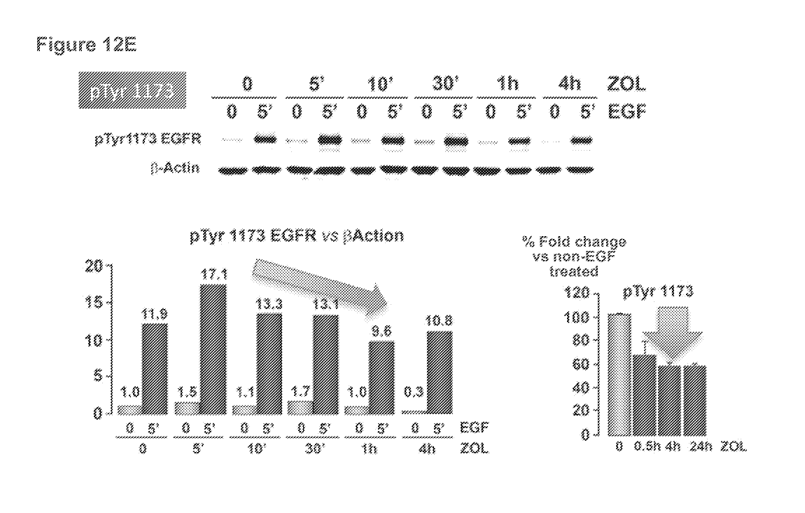

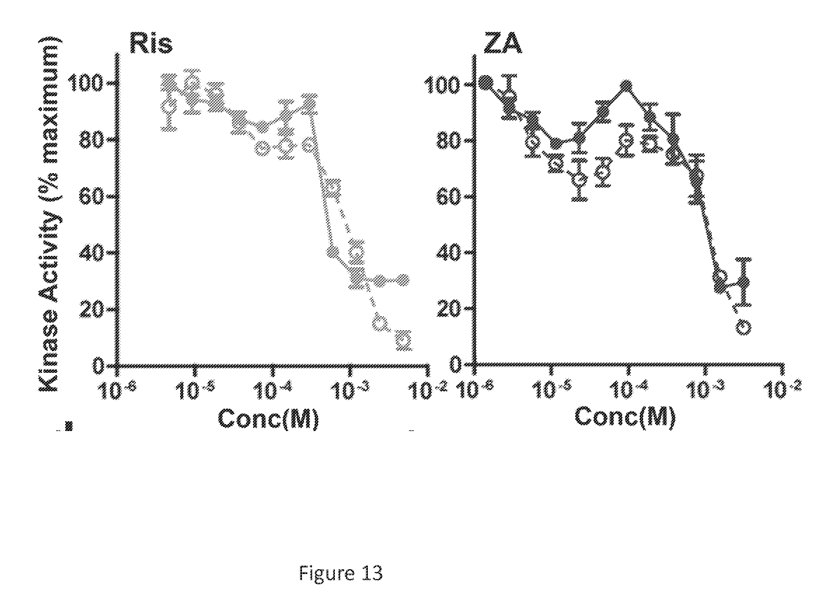

Most cellular actions of bisphosphonates have been shown to occur via alterations in the mevalonate pathway. We used a cell-free in vitro kinase assay using recombinant EGF receptor (EGFR) to determine whether bisphosphonates inhibit the EGFR directly or indirectly. To do so, we incubated the reconstituted, recombinant EGFR with alendronate (ALN) or risedronate (RIS) (100 μM) for 1 hour. A kinase reaction was stimulated with the addition of ATP, following which phosphorylation of the substrate poly(Gly,Tyr) was detected using a horseradish peroxidase (HRP)-conjugated anti-phosphotyrosine antibody. We found that both alendronate and risedronate directly inhibited EGFR kinase activity (FIG. 11). In parallel, a similar cell-free in vitro assay using recombinant EGFR was used to measure the phosphorylation of the EGFR following addition of ATP in the presence or absence of zoledronic acid (ZA, 100 μM). Western immunoblotting using antiphosphotyrosine antibodies to Tyr1045, Tyr1068 and Tyr1173 demonstrated that zoledronic acid inhibited the activation of recombinant EGFR in vitro (FIG. 12). Importantly, this inhibition was not reversed in high [Mg2+] (8 mM), reinforcing direct binding of bisphosphonates, as opposed to an indirect effect via Mg2+ chelation (FIG. 13). Together, the evidence suggests that bisphosphonates directly interact with the EGFR and inhibit its activation.

To understand how bisphosphonates inhibit the EGFR, we docked the bisphosphonates in the crystal structure of human EGFR (PDB id 2GS7) (Zhang, et al., Cell 125, 1137-1149 (2006)). To do so, we built the molecular structures of the bisphosphonates using the Schrödinger Suite 2011 Build module and then minimized these structures with the premin tool. LigPrep module was used to create tautomers, and stereoisomers, and Epik module to generate ionization states at a pH range of 7±1. The less probable states for each molecule, according to the state penalty value, were removed. Different conformations were generated with the ConfGen module. About 650 structures were obtained and then submitted to the docking phase. The EGFR crystal structures in complex with AMP-PNP and Ert was downloaded from the ProteinDataBank (PDB id 2GS7 and 1M17) and prepared with the Schrödinger Suite 2011 ProteinPreparationWizard module. The coordinates of the Mg²⁺ and the water molecule that contributes to its coordination shell were placed between D855 and N842 similarly to that observed in EGFR/AMP-PNP structure (PDB id 2GS7). The essential water that bridges the interactions between AMP-PNP/Ert and residues T790 and T854 was also retained. The missing side chains and hydrogen atoms in the protein were added and optimized with an exhaustive sampling. Finally, the proteins were minimized until a final RMSD of 0.3 Å with respect to the input protein coordinates.

Docking studies were performed with Glide v5.7. Energy grids were built with a van der Waals radius scaling factor of 0.8 and a partial atomic charge less than 0.25. In the docking of the ZA and other bisphosphonates alone, the enclosing and bounding box sizes were set to 8 and 10 Å and centered between the Mg²⁺ ion and the bridging water molecules. In presence of Ert, the grid sizes were set to 8 and 5 Å and centered between Mg²⁺ and Ert. A metal and an H-bond constraint was used to filter poses in which the ligand can interact with the Mg²⁺ ion and make an H-bond with the water molecule (in absence of Ert) or the NH group between the quinazoline and the benzene rings of Ert. The SP docking protocol with default values was used. The two important water molecules along with Mg²⁺ and ERT were treated as integral parts of the protein during the docking phase. For each ligand, the best-scored binding mode, according to Glidescore, was chosen.

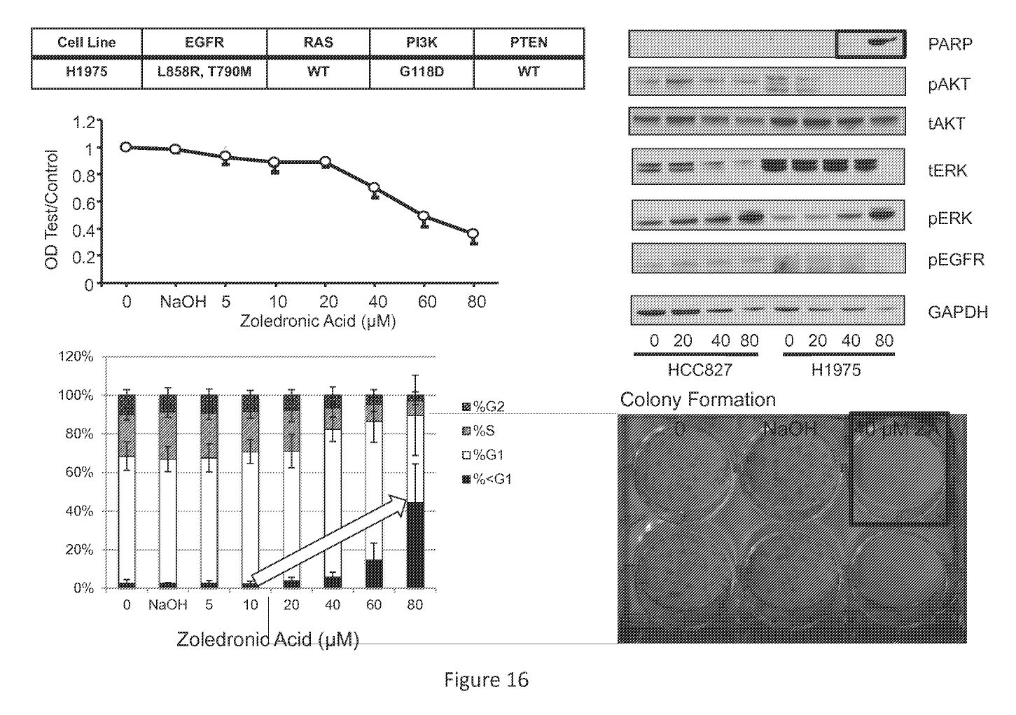

We observed that nitrogen-containing bisphosphonates, including zoledronic acid, could bind into the ATP-binding site of the EGFR kinase domain (FIGS. 16A and B). Notably, the phosphate groups of the bisphosphonate, together with EGFR residues D855, N842 and a water (WAT) molecule, were predicted to coordinate the Mg²⁺ ion, thus mimicking phosphate binding of the nonhydrolysable ATP analog, AMP-PNP. This correlates with the known ability of bisphosphonates to chelate divalent cations. The imidazole ring of zoledronic acid was also predicted to interact with the “gatekeeper” residue T790 and residue T854 via a structural water molecule, which is essential for the binding of ATP, as well as the EGFR inhibitors erlotinib and gefitinib (PDB ids 1M17 and 2ITY, respectively) (Stamos, et al., J Biol Chem 277, 46265-46272 (2002) and Yun et al., Cancer Cell 11, 217-227 (2007)). Superimposition of zoledronic acid onto the crystallized AMP-PNP showed that its imidazole ring overlaps with the purine ring of the adenine moiety of ATP. This demonstrates that zoledronic acid can compete with ATP, but would be expected to have a weaker affinity since it lacks interactions with the hinge region of the EGFR.

### Example 3

### Bisphosphonates Selectively Kill EGFR-Positive Cancer Cells

Connectivity mapping, cell-free assays, and computational modeling together suggest that the EGFR is a target for bisphosphonate action. To validate this, we evaluated the cellular effects of bisphosphonates in EGFR-negative and EGFR-positive cancer cell lines. Mutated cancer cell lines, namely H520, H1666, H3255, HC827, A549, H1650, MCF-7, Caco-2, SW620, and SW480, were grown using standard protocols, and exposed to bisphosphonates. Thereafter, the cells were subject to the MTT assay to assess viability using a commercial kit. Additionally, in certain instances, annexin V staining was performed using a previously described protocol. Quantitative PCR (qPCR) for BCL, Bax, survivin, as well as for the thirteen bisphosphonate signature genes (FIG. 1) was carried out. Protein extracts were subject to SDS-PAGE followed by Western immunoblotting using antibodies to PARP, Akt (total and phosphorylated), Erk (total and phosphorylated), cyclin D1, cyclin B1, PCNA, and GAPDH (Santa Cruz, USA). Flow cytometry and colony forming assays were also performed.

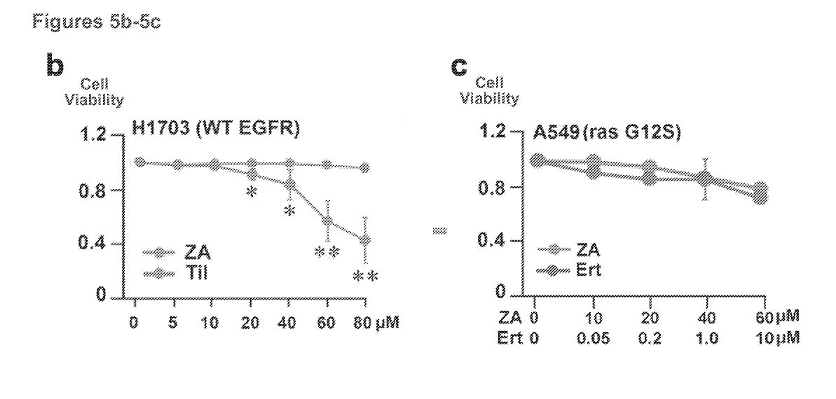

In the EGFR-negative lung cancer cell line H520, zoledronic acid displayed minimal or no effects on cell viability or apoptosis, or on the expression of apoptosis and survival genes when exposed for up to 72 hours (FIG. 5A). Likewise, the EGFR-low breast cancer cell line MCF-7 failed to respond to zoledronic acid (FIG. 5A). However, cell survival was significantly attenuated in the Caco-2 colon cancer cell line and in H1666 lung cancer cells that express the wild type EGFR at moderate and high levels, respectively (FIG. 5B). In contrast, a lung cancer cell line A549 that expresses wild type EGFR, but is driven by a Gl2S ras mutation, was poorly responsive to both zoledronic acid and erlotinib (Ert) (FIG. 5C). These data suggest that only tumors driven by EGFR signaling (either by mutation or upregulation of EGFR) are sensitive to bisphosphonates.

About 15% of all non-small cell lung cancers are driven by mutations that render EGFR constitutively active, resulting in oncogenic dependence of these tumors and an overall poor prognosis. The most common mutations reside in the EGFR kinase domain, and include a missense (L858R) and deletion mutation (746-750). As observed in X-ray crystal structures of wild type and L858R EGFR, the protein can adopt different conformational states. In the inactive state of wild type EGFR, the Cα-helix is positioned in an outward conformation with residue M766 located at the base of the helix in the hydrophobic pocket beyond the gatekeeper residue T790. Residues immediately after the “DFG motif” in the activation loop (L858 onwards) form a short helix, which prevents the collapse of Cα-helix onto the binding site. In the active state of the wild type EGFR, the short helix uncoils resulting in a change in conformation of the activation loop (PDB id 2GS6). This allows the Cα-helix to collapse towards the binding site and activate the kinase, forming the catalytically important KE salt bridge between K745 and E762. The conformational state of L858R mutant (PDB id 2ITV) resembles that of the active state. Here, R836 forms a salt bridge with E866, and this ion pair helps to neutralize the positive charge repulsion effect between R836 and the mutated residue R858. Meanwhile, R858 makes a hydrogen bond with Y891. This interaction pulls the activation loop and allows the Cα-helix to collapse towards the binding site, resulting in constitutive EGFR activation. This is confirmed by molecular dynamic analysis, which shows that the interaction between L858R-Y891 locks the activation loop in a conformation that allows Cα-helix to collapse and adopt an activated conformation in the presence of ATP.

As with the wild type receptor, zoledronic acid and erlotinib could both bind to the adenine-binding domain of the L858R mutant. Anisotropic network modeling (ANM) was therefore utilized to examine conformational changes over microseconds induced upon such binding. ANM demonstrated that the Cα-helix collapsed despite the presence of erlotinib. This suggests that the known increases and decreases in affinity of the L858R mutant to erlotinib and ATP, respectively, are independent of the movement of the Cα-helix. Likewise, irrespective of the position of the Cα-helix, zoledronic acid binding to the L858R mutant may inhibit downstream signaling, as noted (FIG. 11), due to the presence of a non-hydrolysable γ-phosphate.

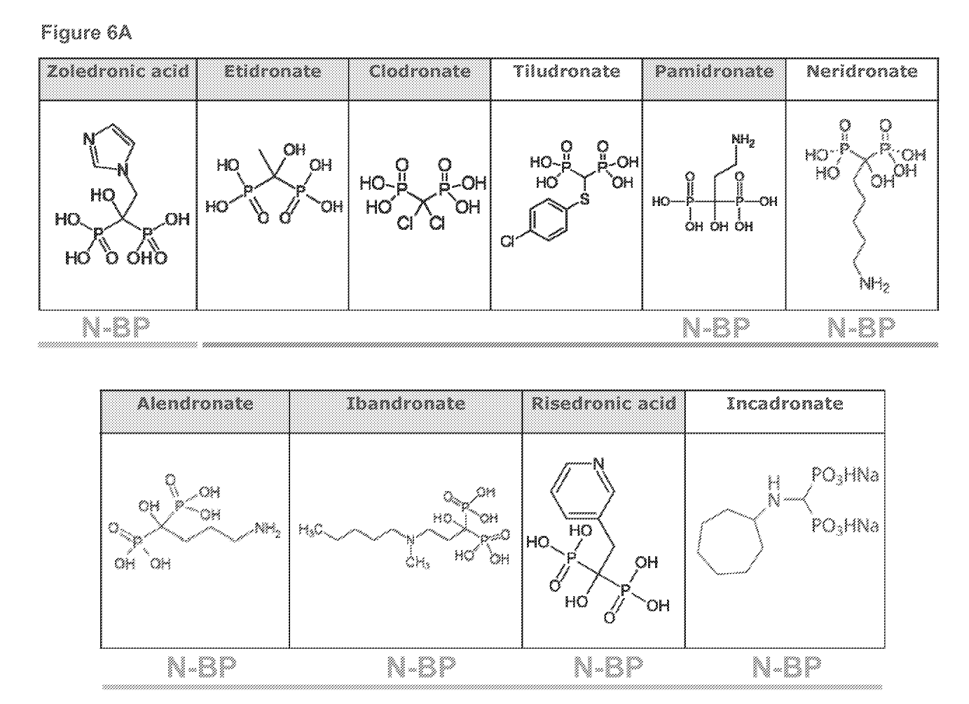

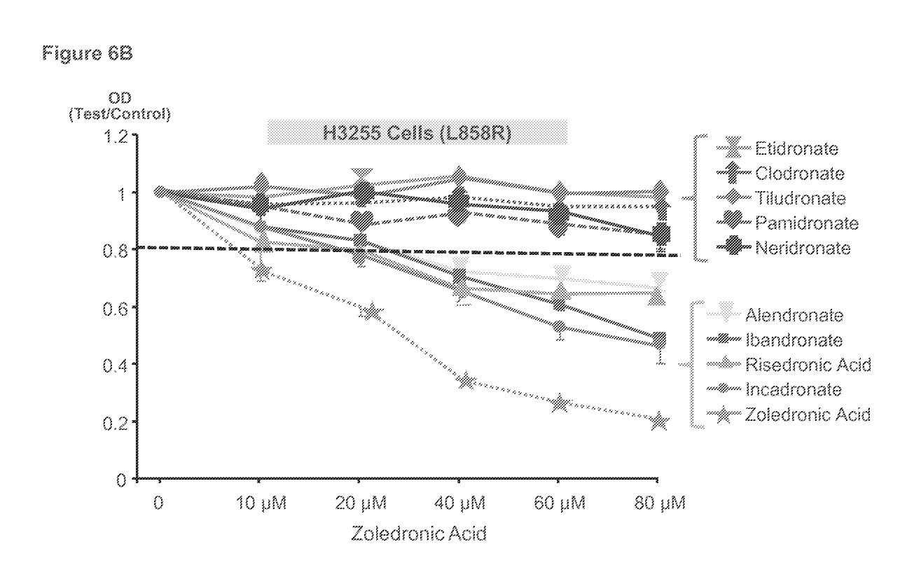

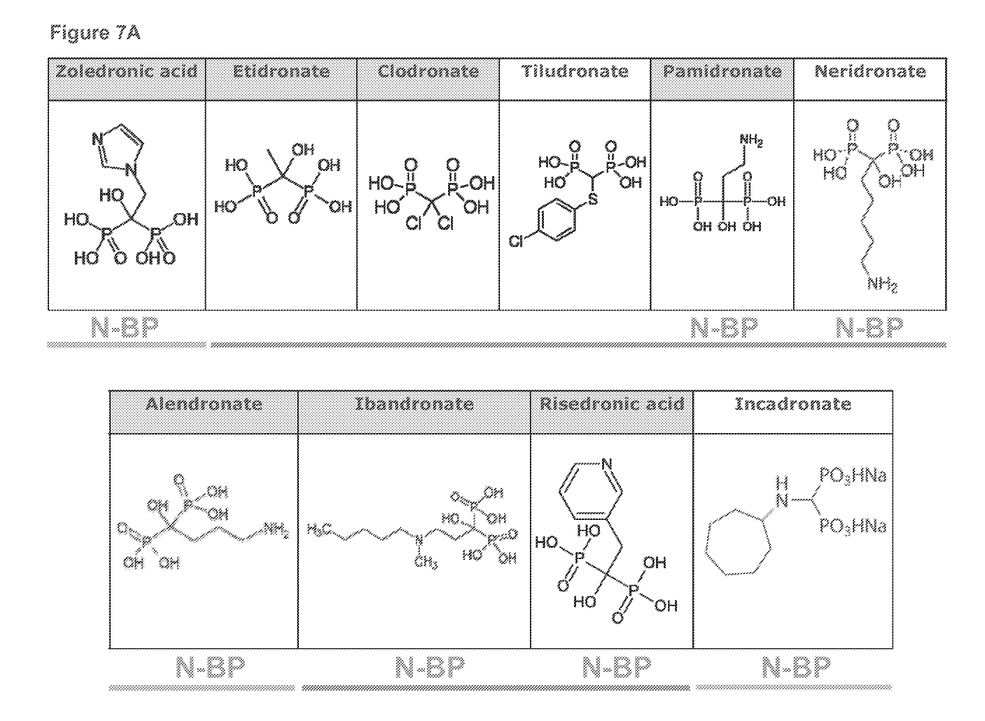

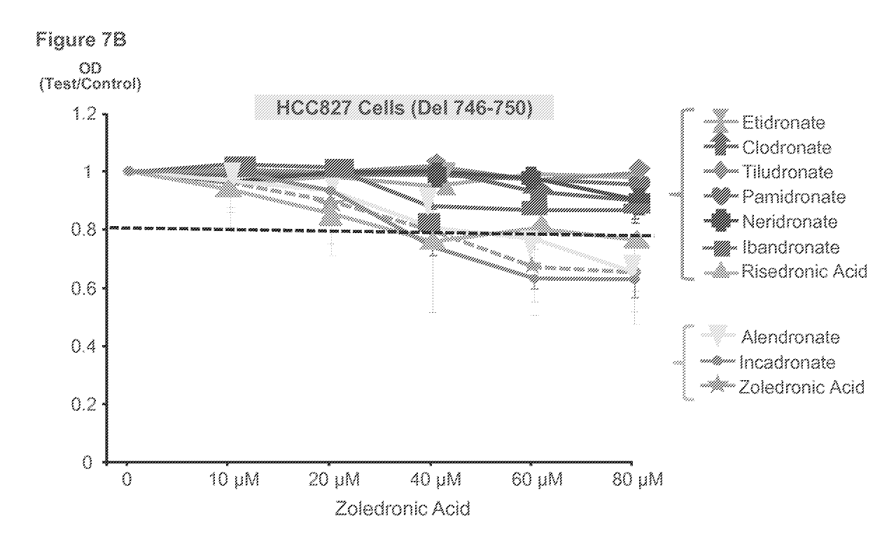

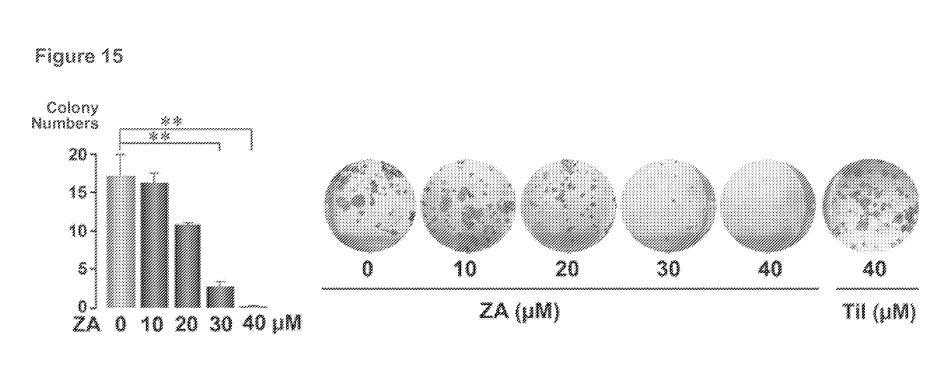

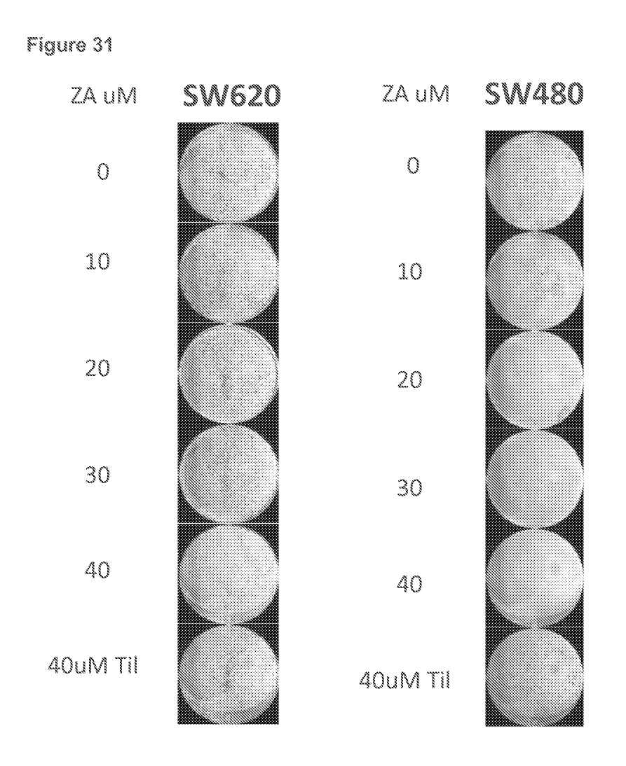

Based on this prediction, and the common occurrence of these mutations in lung cancer patients, we examined the effect of available approved bisphosphonates on lung cancer cell lines that are driven by the L858R (H3255) or del746-750 (HCC827) mutation (FIGS. 6 and 7). The bisphosphonates, notably zoledronic acid, minodronic acid, risedronic acid, alendronate and incadronate, displayed a rank order of potency in inhibiting cell survival in both cell lines (FIGS. 6, 7 and 14). Each bisphosphonate capable of inhibiting cell survival contained a nitrogen atom. In contrast, the non-N-containing bisphosphonates, such as tiludronate, failed to affect cell viability. The effect of zoledronic acid on cell viability was mirrored by a marked concentration-dependent inhibition of colony formation in HCC827 cells, which was not seen with tiludronate (40 μM) (FIG. 15). Further zoledronic acid reduced colony formation in EGFR-expressing colon cells (SW480), but had no effect on EGFR negative (SW620) colon cancer cell line. The non-N-containing bisphosphonate tiludronate has no effect on either of the colon cancer cell lines tested (FIG. 31). Western analysis revealed that zoledronic acid induces cell cycle arrest in SW480 but not SW620, as evidenced by decreased cyclin D1 protein (data not shown).

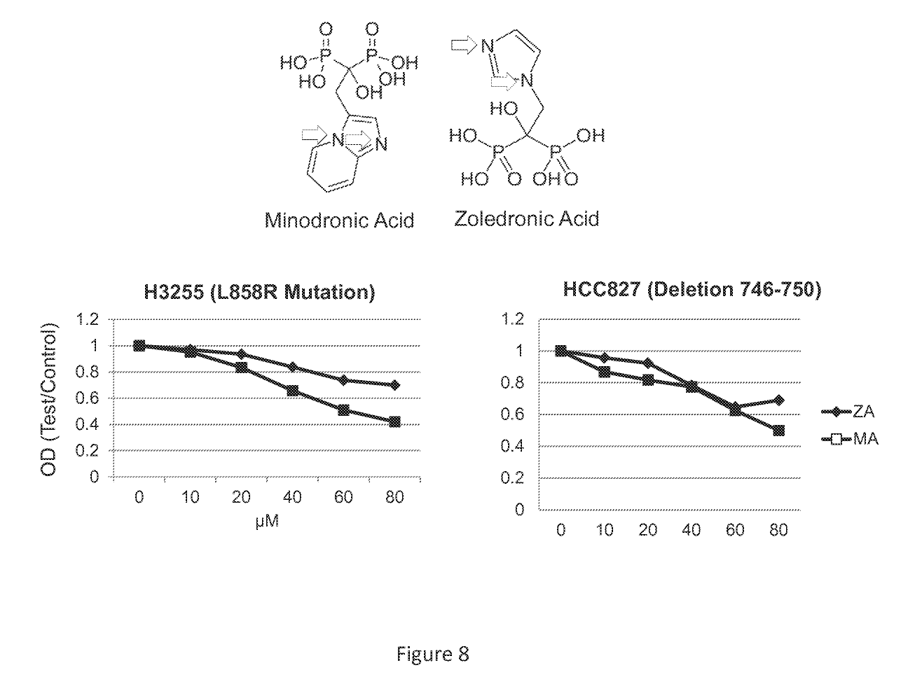

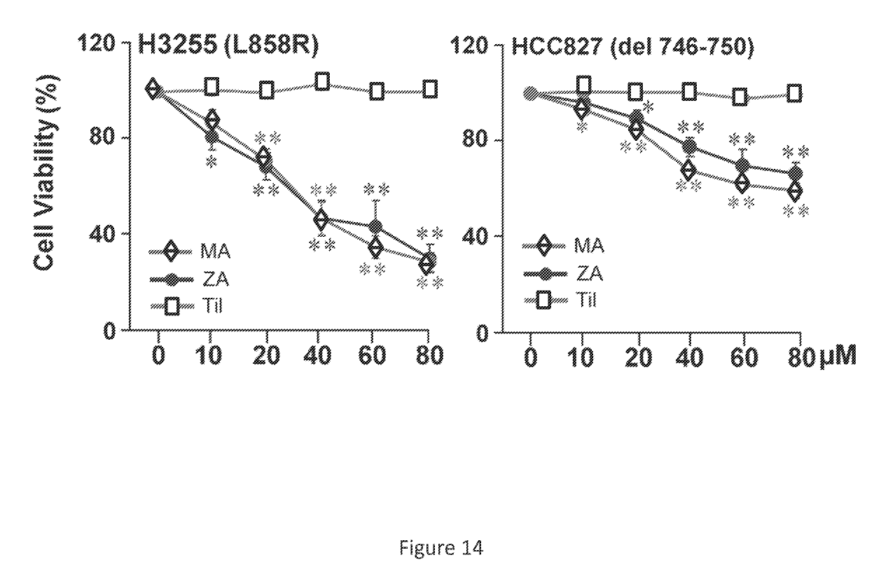

Zoledronic acid and minodronic acid were the two most potent bisphosphonates in reducing the viability of EGFR mutation-positive cancer cells (FIG. 14). In contrast, tiludronate was without effect on either cell line (FIG. 14). Zoledronic acid and minodronic acid have a five member imidazole ring that resembles the imidazole-containing purine ring of ATP, whereas tiludronate possesses a p-chlorophenyl ring without a nitrogen atom (FIGS. 6, 7, and 8). Molecular docking revealed that while the imidazole rings of zoledronic acid and minodronic acid interact with T790 of EGFR through a water molecule, the p-chlorophenyl ring of tiludronate does not. The phosphate groups of both molecules could, however, coordinate with the Mg²⁺ ion, as noted with zoledronic acid. Without being bound by theory, these data suggest that Mg²⁺ chelation cannot in itself explain the inhibitory actions of the N-bisphosphonates, and that effective bisphosphonates must interact with T790 and bind to the adenine-binding site to displace ATP. These observations are consistent with the fact that not all bisphosphonates with phosphate residues reduce cell viability.

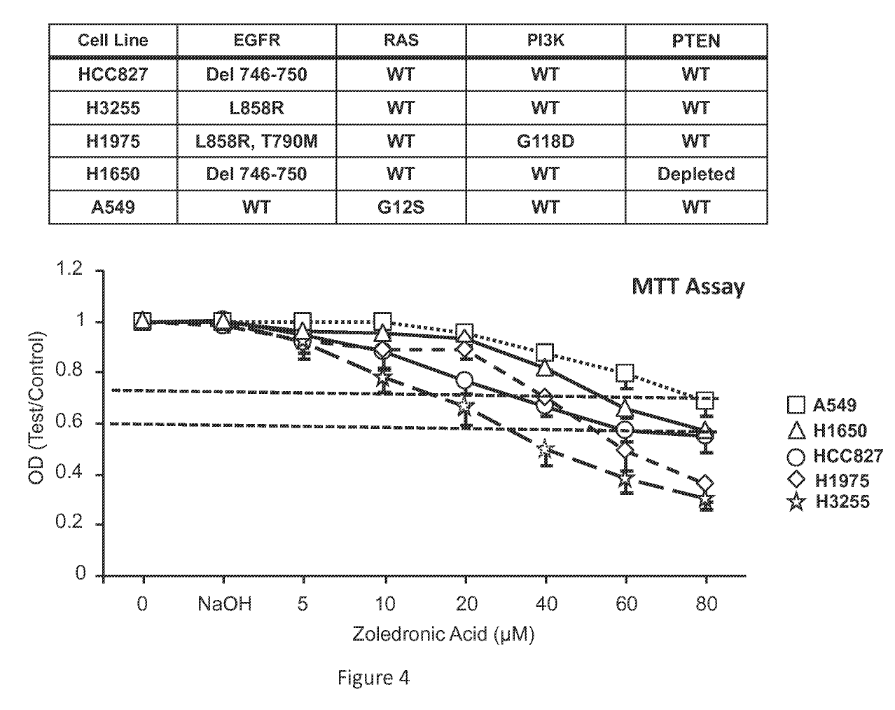

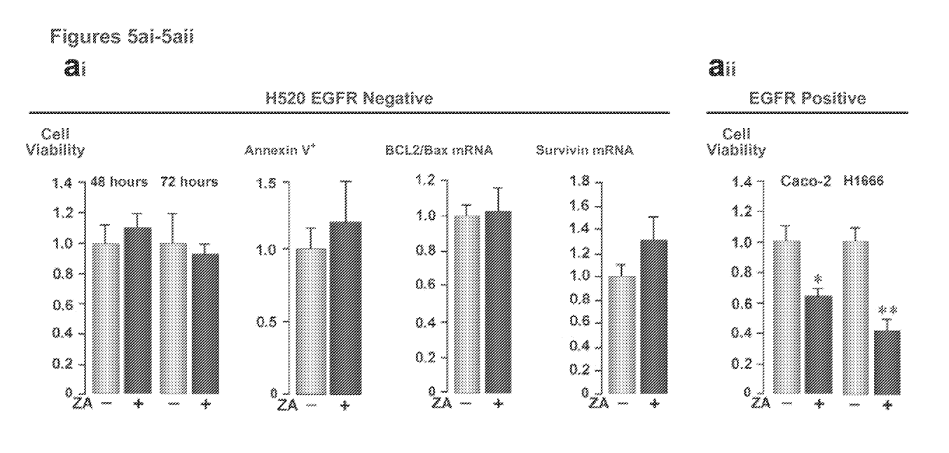

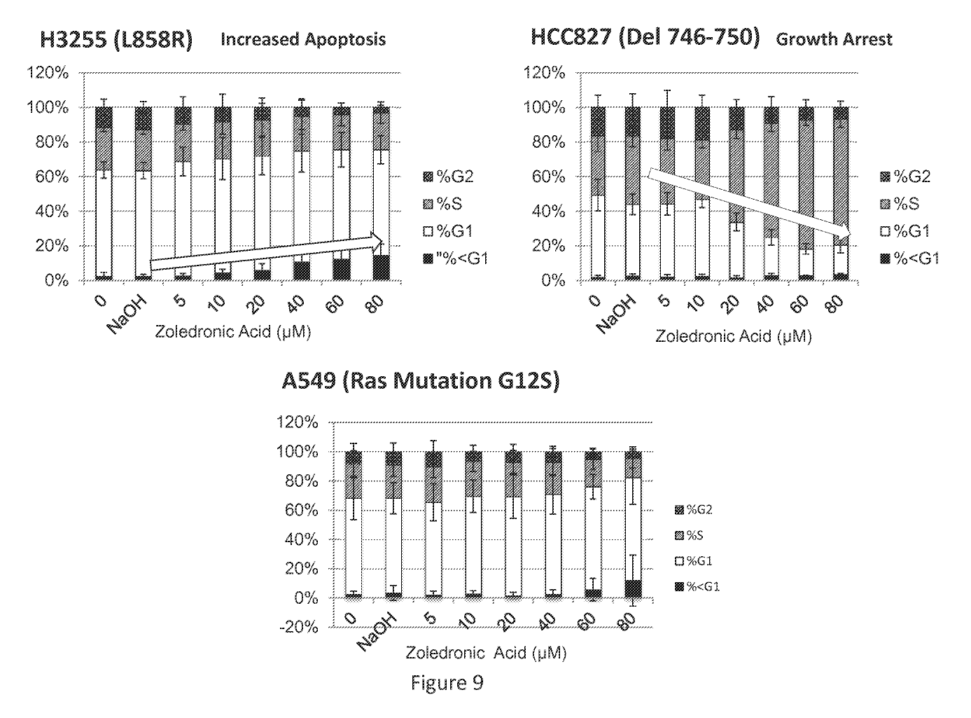

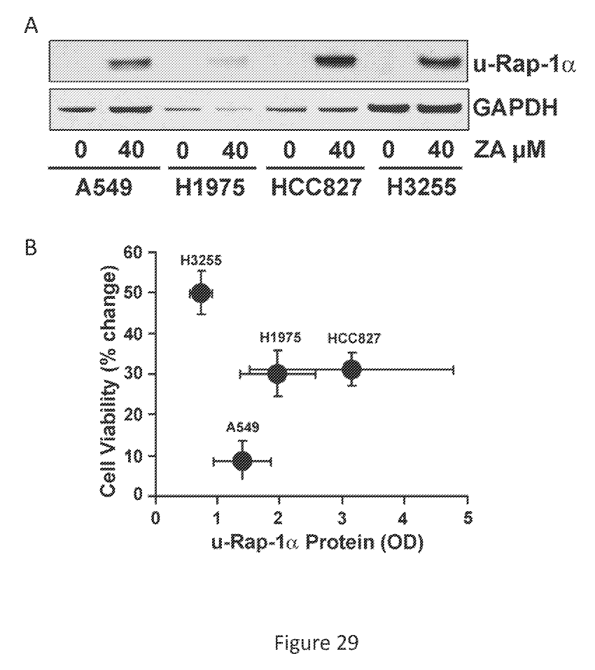

Without being bound by theory, the data also suggest that farnesyl diphosphate synthase (FPPS) inhibition does not contribute significantly to the effect of bisphosphonates on EGFR mutation-positive lung cancer cells. First, while the pamidronate and neridronate are known to inhibit FPPS and to reduce bone resorption, they failed to affect HCC827 cell viability (FIG. 7). Furthermore, expected from its inhibition of FPPS, zoledronic acid caused the accumulation of unprenylated Rab1, importantly, to an equal extent in H3255, HCC827 and A549 cells (FIG. 29). Yet, ras-driven A549 cells displayed a poor response to zoledronic acid compared with H3255 or HCC827 cells (FIGS. 4 and 9). Thus, the extent of FPPS inhibition by bisphosphonates does not appear to match their effect in reducing the viability of EGFR mutation-positive lung cancer cells. We cannot, however, exclude the possibility that the anti-apoptotic actions of bisphosphonates are in part mediated through this pathway.

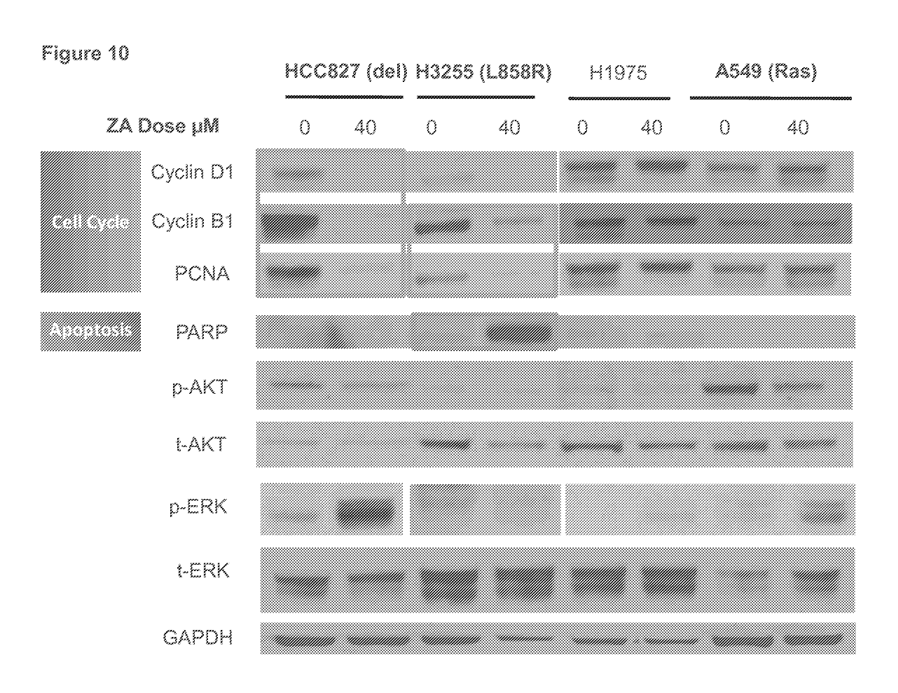

To examine the underlying cellular mechanism through which bisphosphonates decrease cell viability, we performed FACS analysis and Western blotting for molecules involved in the cell cycle and apoptosis. HCC827 cells showed a dramatic, concentration-dependent cell cycle arrest (FIG. 9), whereas H3255 cells displayed a significant increase in apoptosis shown by enhanced PARP expression (FIG. 10). A reduction in cyclin B1, cyclin D1, and PCNA expression in both cell lines (FIG. 10) was also observed. The minimally responsive cell line A549 showed apoptosis and increased PARP expression only at the highest, 80 μM, zoledronic acid concentration (FIG. 10 and data not shown). Thus, bisphosphonates appear to reduce cell viability through a combination of effects on apoptosis and the cell cycle. These data further suggest that cancers must be dependent upon EGFR activation to be bisphosphonate sensitive.

### Example 4

### Bisphosphonates Synergize the Apoptotic Effects of EGFR Kinase Inhibitors

After showing that bisphosphonates can act as EGFR inhibitors, we investigated the possibility that bisphosphonates may synergize the action of the existing EGFR kinase inhibitors erlotinib and gefitinib, and thus offer a greater therapeutic advantage over either agent alone. To determine whether the two drug classes can bind simultaneously in the EGFR kinase pocket to affect receptor function, we performed a combination of molecular docking, MD and ANM analyses. The wild type EGFR crystal structure in complex with erlotinib (PDB 1M17) was used as the basis for the analyses. A Mg²⁺ ion was placed between D855, N842 and a water molecule in a similar position to that observed in the EGFR/AMP-PNP crystal structure (PDB id 2GS7). Erlotinib and Mg²⁺ were treated as integral parts of the protein during the docking phase. We found that that there was enough space in the pocket to allow for the simultaneous binding of erlotinib and zoledronic acid. Erlotinib binds primarily by replacing the adenine ring of ATP. In contrast, the phosphates of zoledronic acid coordinate the Mg²⁺ ion, mimicking the binding of AMP-PNP. Zoledronic acid also forms a hydrogen bond with the NH group between the phenyl and quinazoline rings in erlotinib, which, in turn, interact with T790 and T854 similarly to AMP-PNP. Similar binding modes were observed for the zoledronic acid/gefitinib and minodronic acid/erlotinib pairs. Importantly, not all bisphosphonates were capable of simultaneous binding with erlotinib or gefitinib. Tiludronate, for example, does not have an imidazole ring to make an H-bond with erlotinib. Instead, tiludronate's p-chlorophenyl group displayed severe steric clashes with the erlotinib molecule.

To examine the impact of the simultaneous binding of zoledronic acid and erlotinib on the conformation of the L858R mutant (PDB 2ITT), we performed ANM and MD in complement. As noted previously, the L858R mutation causes a collapse of the Cα-helix onto the binding site rendering the EGFR constitutively active. Both ANM and MD showed that, in the presence of erlotinib and zoledronic acid, the Cα-helix locks in a collapsed conformation, which becomes similar to that of active wild type EGFR in the presence of ATP. However, due to the known ability of erlotinib to displace ATP and the presence of a non-hydrolysable γ-phosphate in zoledronic acid, the two drugs together were predicted synergize in preventing downstream phosphorylation.

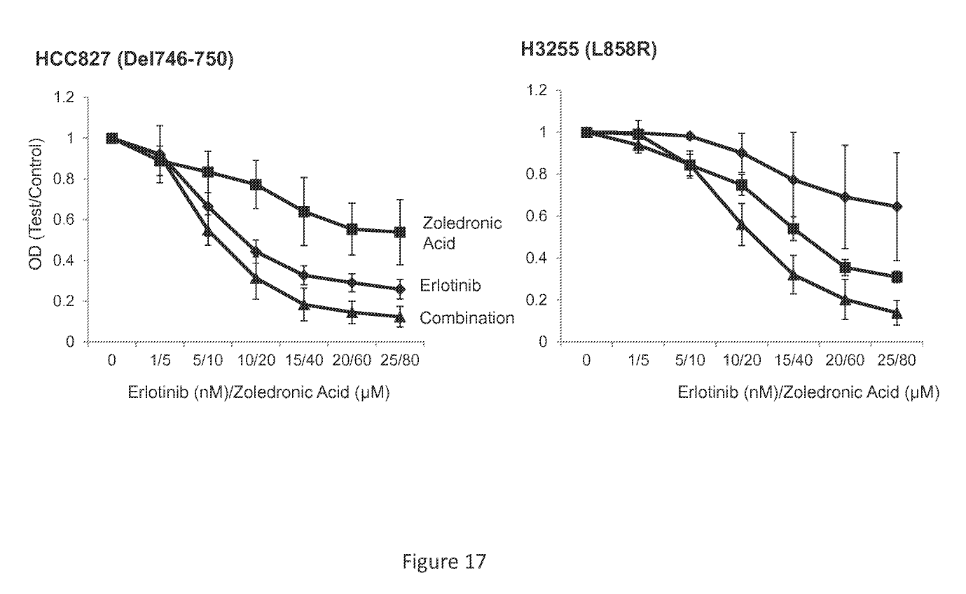

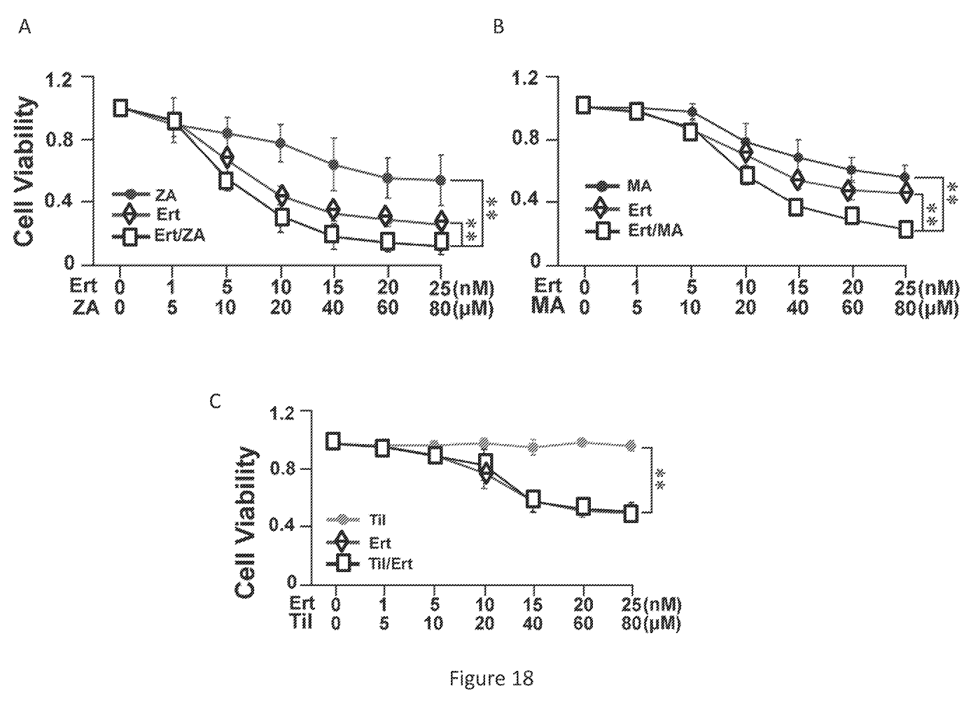

When a sub-maximal concentration of zoledronic acid (10 μM) was combined with a sub-maximal concentration of erlotinib (5 nM), the inhibition of colony formation in HCC827 cells was significantly greater than with either drug alone (FIG. 21). The bisphosphonate-specificity of the observed synergy was investigated in cell viability assays. The effect of erlotinib was significantly enhanced when the drug was combined with either one of the imidazole-containing bisphosphonates zoledronic acid or minodronic acid (FIGS. 17 and 18). However, when erlotinib was combined with tiludronate, which does not have an imidazole ring, the synergistic inhibition was not observed (FIG. 18). These data agree with the above modeling studies predicting simultaneous binding of erlotinib with zoledronic acid or minodronic acid, but not with tiludronate.

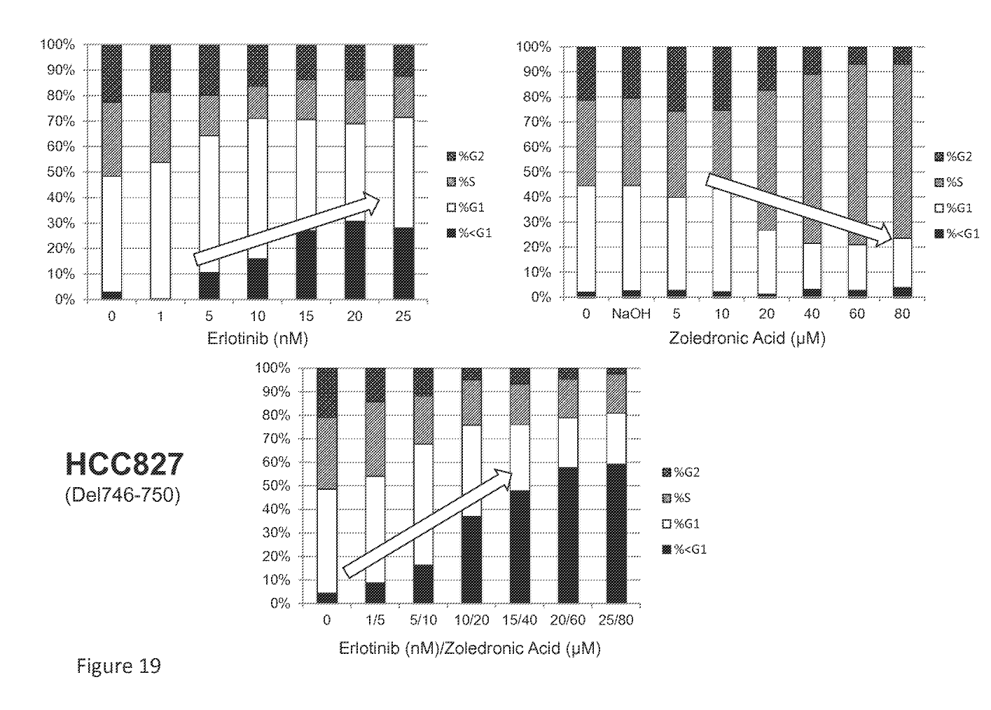

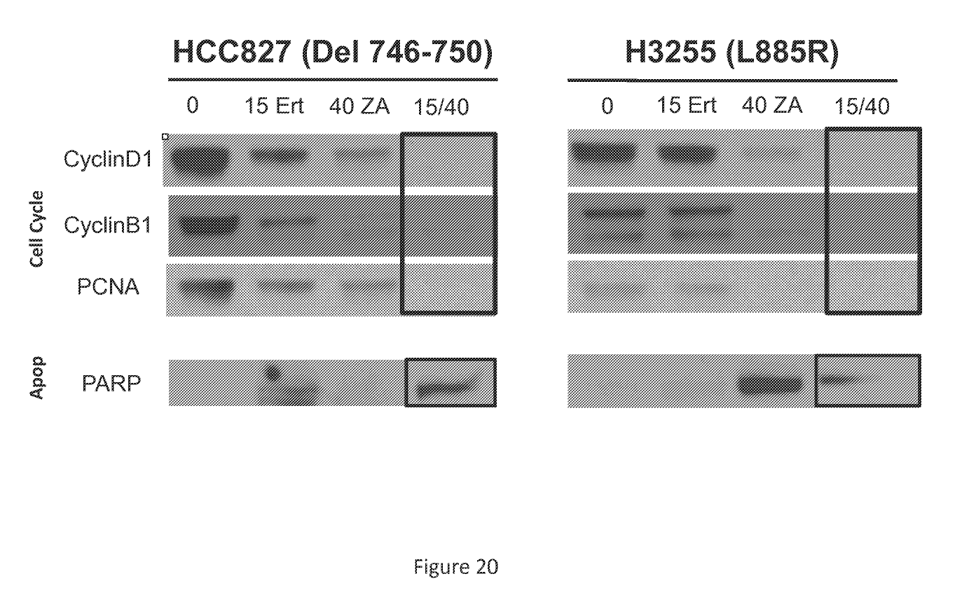

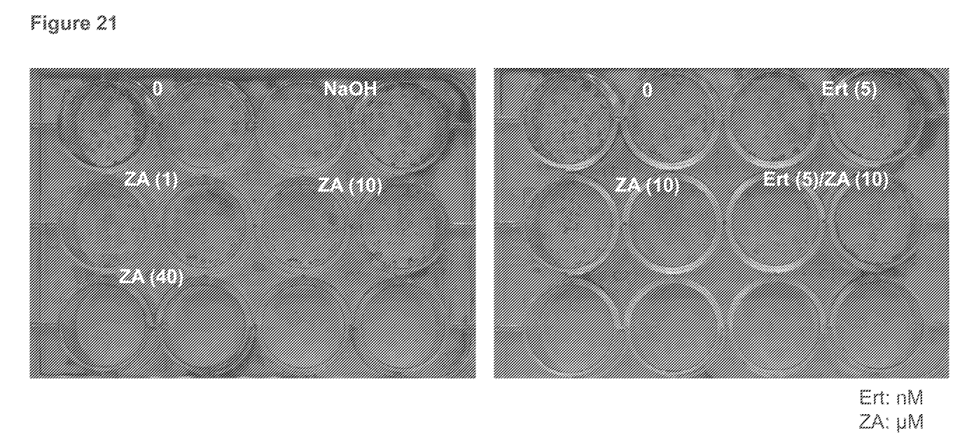

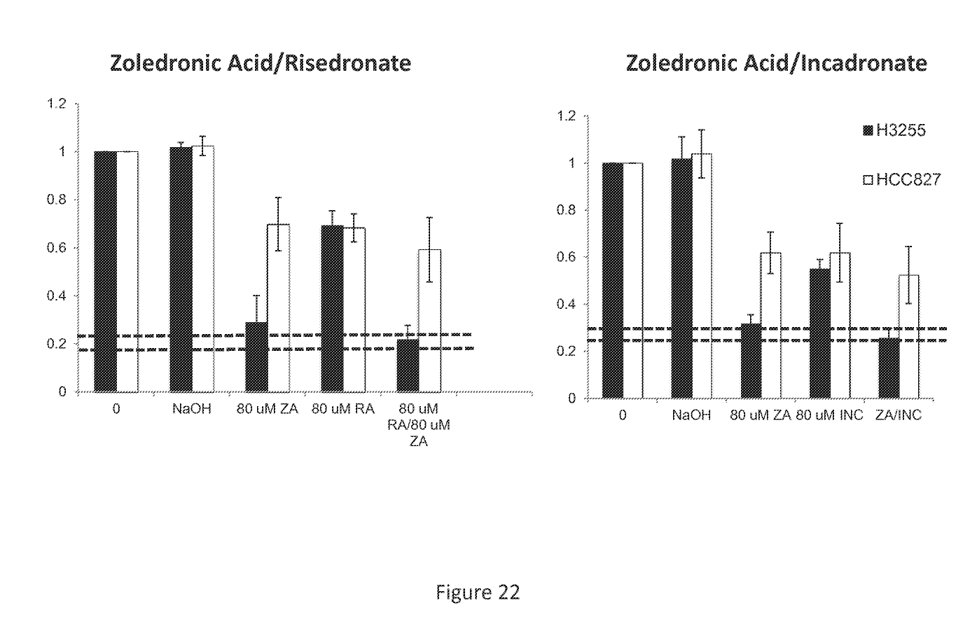

The profound decrease in cell viability from combining zoledronic acid and erlotinib was due to a marked increase in apoptosis, as shown by FACS analysis and Western blotting (FIGS. 19 and 20). Whereas erlotinib mainly increased PARP expression and apoptosis, and zoledronic acid primarily caused cell cycle arrest, the two together triggered a greater change in apoptosis, PARP expression, and cyclin B1/D1 expression than either agent alone. This remarkable synergy noted between zoledronic acid and erlotinib was not seen on combining two N-containing bisphosphonates, namely zoledronic acid and risedronate or zoledronic acid and incadronate (FIG. 22). Without being bound by theory, these data suggest that synergistic EGFR inhibition requires two drugs that interact with distinct binding modes.

### Example 5

### Bisphosphonates Overcome the Resistance to Erlotinib Induced by the Gatekeeper Mutation T790M

The EGFR inhibitors erlotinib and gefitinib induce significant levels of tumor death and trigger rapid and durable clinical responses in EGFR mutation-positive lung cancers. Invariably, however, resistance to therapy develops with the most common mechanism being the acquisition of a second site mutation at the gatekeeper residue T790. The T790M mutation (PBD id 2JIU) causes only a partial collapse of the Cα-helix as mutant M790 acts as a wedge and interferes with the spatial positioning of the side chain of the highly conserved M766. Movement of the activation loop region observed on MD shows that the Cα-helix assumes an activated conformation, with the T790M mutation preventing a complete collapse of the helix over the binding site.

Resistance to erlotinib/gefitinib arises from increased affinity for ATP rather than lower affinity of the respective inhibitors. This is thought to be due to altered interactions between M790 and M793, although the precise molecular events are unclear. In contrast, the binding mode of the imidazole ring of zoledronic acid was not affected by the T790M mutation. Specifically, the water molecule that bridges the interaction of zoledronic acid with M790 and T854 is preserved. Thus, while the T790M mutation reduces sensitivity to erlotinib/gefitinib, sensitivity to zoledronic acid is not affected by this mutation.

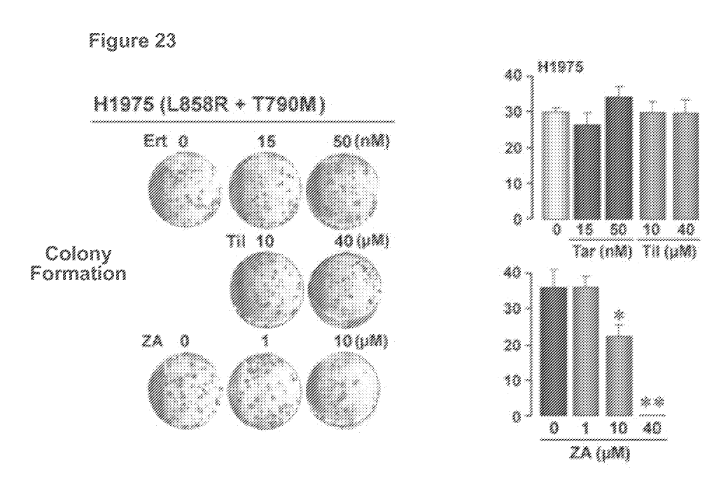

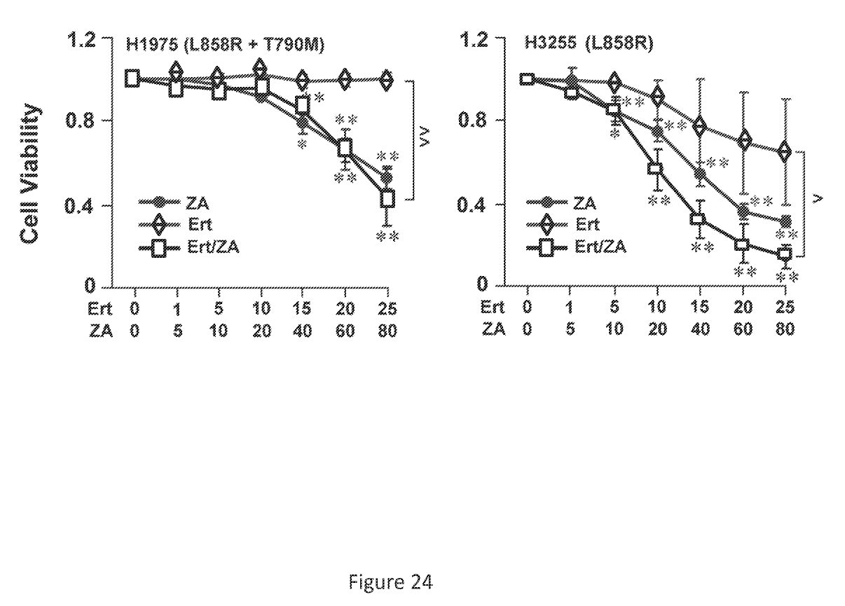

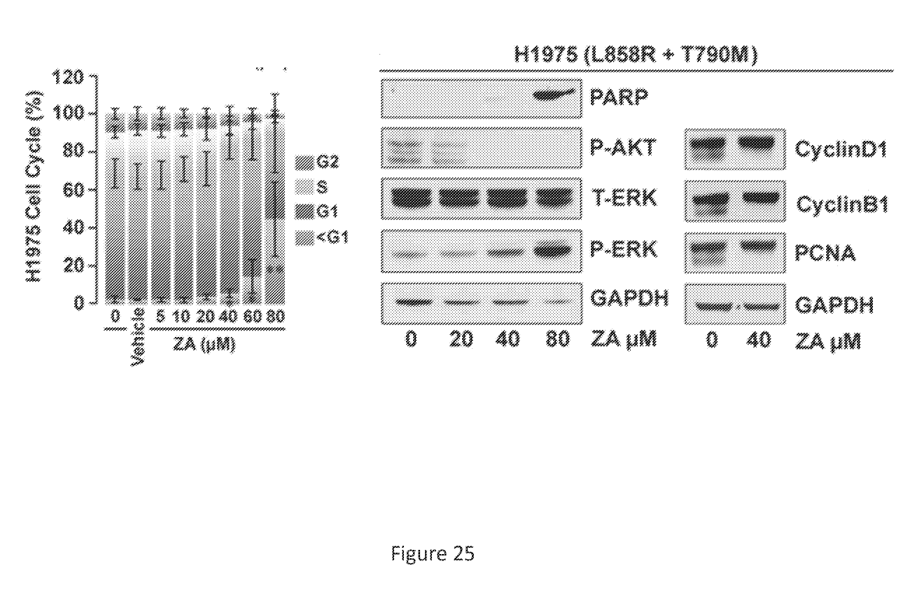

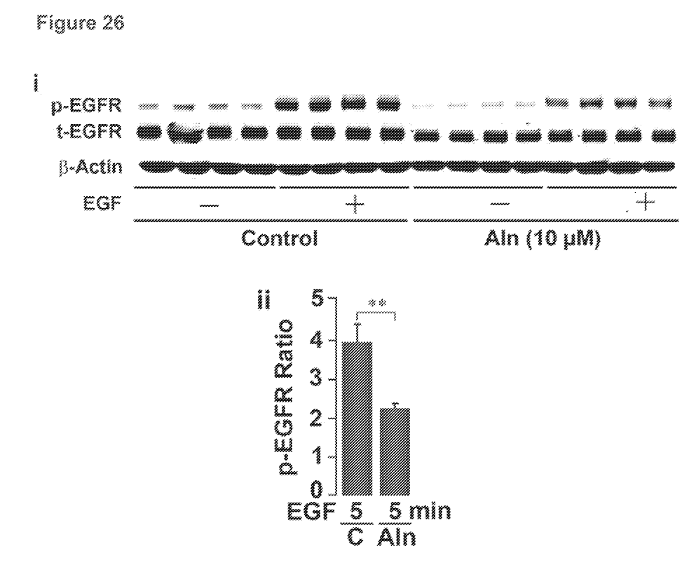

To test this hypothesis, colony formation and cell viability assays were carried out using a L858R/T790M-derived lung adenocarcinoma cell line, H1975. Whereas erlotinib did not inhibit colony formation, zoledronic acid did (FIG. 23). Tiludronate, which did not inhibit cell viability (c.f. FIG. 14), also did not affect colony formation in H1975 cells. Consistent with this, cell viability assays showed a concentration-dependent inhibition by zoledronic acid, but not by erlotinib (FIG. 24). This effect is quite distinct from that seen with the cell line H3255 (L858R) that does not harbor the gatekeeper mutation T790M (FIG. 24). The inhibitory effects of zoledronic acid were exerted through a reduction in EGFR autophosphorylation and the stimulation of apoptosis, as evident from sharp increases in PARP expression and reduced Akt phosphorylation (FIG. 25). In contrast to HCC827 (del746-750) cells (FIG. 10), there were minimal changes in the expression of cell cycle proteins in the double mutant cells (FIG. 25).

Importantly, erlotinib did not interfere with the effect of zoledronic acid, so that the effect of the two molecules together was identical to that of zoledronic alone (FIG. 24). This was confirmed on ANM, which showed that both molecules can indeed bind to the double mutant, L858R/T790M. Together, the data underscore the idea that both drugs can be used simultaneously in patients with the gatekeeper mutation T790M, without erlotinib interfering with zoledronic acid action.

### Example 6

### Bisphosphonates Synergize with EGFR Kinase Inhibitors In Vivo

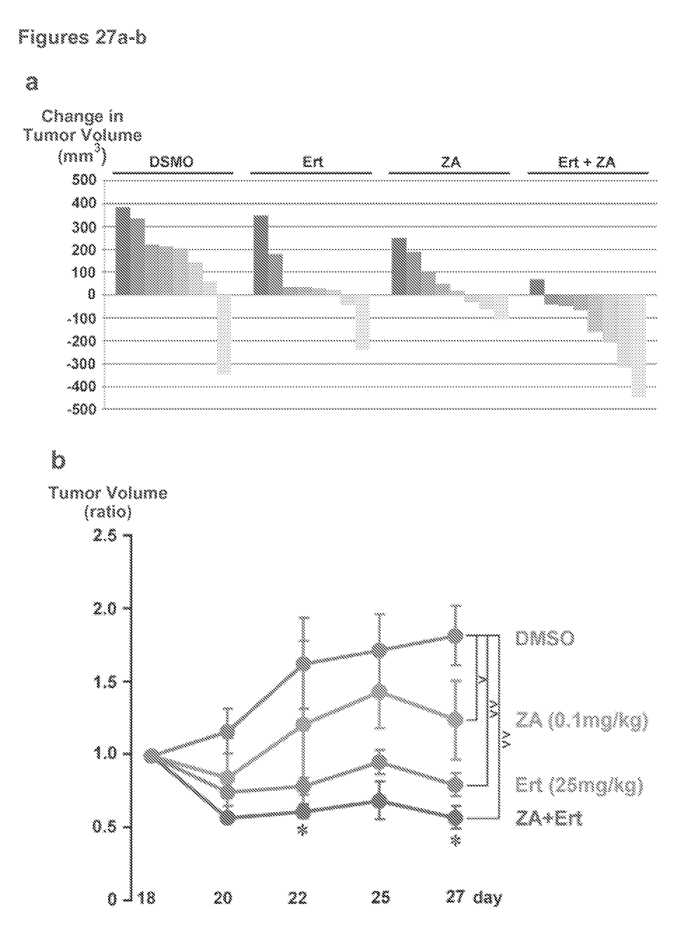

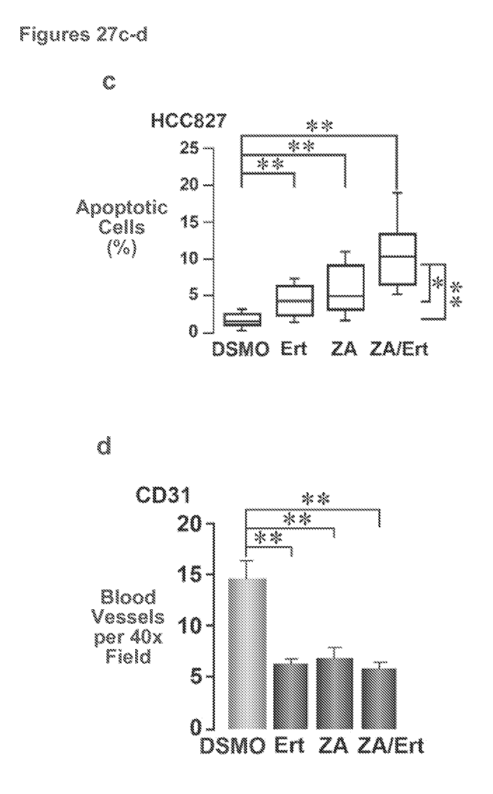

To verify that that the in vitro experiments described above translated to in vivo efficacy, the effect of bisphosphonates and EGFR inhibitors on tumors in mice were evaluated. HCC827 cells (1×10⁸) were injected into the right flank of 6- to 8-week-old female BALB/c nu/nu mice. Tumor volume was assessed weekly until volumes reached an average of 300 mm³, at which time mice were randomized into four treatment groups: vehicle control (DMSO), erlotinib, zoledronic acid, and erlotinib in combination with zoledronic acid. Erlotinib (25 mg/kg) was injected intraperitoneally and zoledronic acid (0.1 mg/kg) was injected intratumorally. A total of 10 treatments were given, with a 48-hour rest period between injections. Tumor volumes were measured during the rest period following each treatment. Erlotinib and zoledronic acid each, individually, slowed tumor growth over the first six or five treatment periods, respectively, before causing a reduction in tumor size (FIGS. 27A and B). The combination of erlotinib and zoledronic acid, however, showed relatively little tumor growth following the first treatment period and caused a dramatic reduction in tumor size beginning in the second treatment period (FIGS. 27A and B). These data confirm that the synergy between bisphosphonates and EGFR inhibitors observed in vitro translates into a synergistic efficacy in vivo.

To verify the in vivo mechanism of action, Western analysis and immunohistochemistry were performed on tumor samples at the end of the 10-treatment course. For Western analysis, whole cell protein extracts were obtained with radio-immunoprecipitation assay buffer following standard protocols (Santa Cruz Biotechnology). The protein extracts were denatured and 40-50 μg, as determined by the Bio-Rad DC Protein quantification assay (Richmond, Calif.), were separated on 12% SDS PAGE gels and transferred to nitrocellulose membranes. After blocking with 5% non-fat milk (Labscientific, Inc.) in TBS-Tween buffer, the membranes were probed with rabbit antibodies specific for p-AKT (no. 4058, Cell Signaling Technology), AKT (no. 9272, Cell Signaling Technology), p-ERK (no. 9272, Cell Signaling Technology), ERK (no. 4695, Cell Signaling Technology), and goat polyclonal actin antibody (sc-1616, Santa Cruz Biotechnology Inc.). For immunohistochemistry, paraffin-embedded tumors were stained with anti-PCNA (FL-261, Santa Cruz Biotechnology Inc.), anti-p-AKT (4060, Cell Signaling Technology), anti-p-ERK (4370, Cell Signaling Technology), and anti-p-EGFR (3777, Cell Signaling Technology). Biotinylated anti-rabbit IgG (E0432, Dako) was used as secondary antibody. Staining was visualized using DAB, and slides were counterstained with hematoxylin, dehydrated, and mounted. Bright-field and fluorescent images were captured using a Stereoscope or Axioplan 2 IE microscope (Zeiss). Both Western analysis and immunohistochemistry demonstrated that erlotinib and zoledronic acid each inhibited AKT phosphorylation, and the combination of erlotinib and zoledronic acid synergized to inhibit AKT phosphorylation (data not shown). Erlotinib alone did not inhibit ERK phosphorylation, while zoledronic acid was able to inhibit ERK phosphorylation. The combination of erlotinib and zoledronic acid, however, synergistically inhibited ERK phosphorylation. Similarly, the combination of erlotinib and zoledronic acid on EGFR phosphorylation was greater than the effect of erlotinib or zoledronic acid alone.

The combination of erlotinib and zoledronic acid also had a synergistic effect on apoptosis (FIG. 27C). Interestingly, while zoledronic acid and erlotinib both inhibited CD31 expression, the effects of the two drugs were not synergistic (FIG. 27D). This suggested that synergy between erlotinib and zoledronic acid is confined solely to tumor cells, and not to CD31-bearing, vascular endothelial cells.

### Example 7

### Effect of Bisphosphonates on Inhibition of HER2

To determine the effect of bisphosphonates on other members of the EGFR-family, we performed modeling studies and in vitro experiments using HER2. Specifically, MD demonstrated that nitrogen-containing rings of risedronate, zoledronic acid and minodronic acid were capable of interacting with T798 of HER2 via a water molecule.

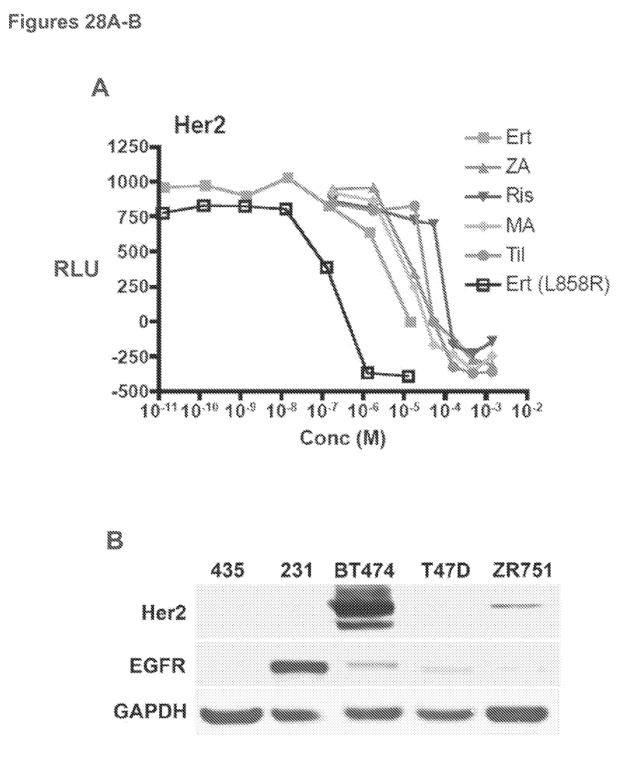

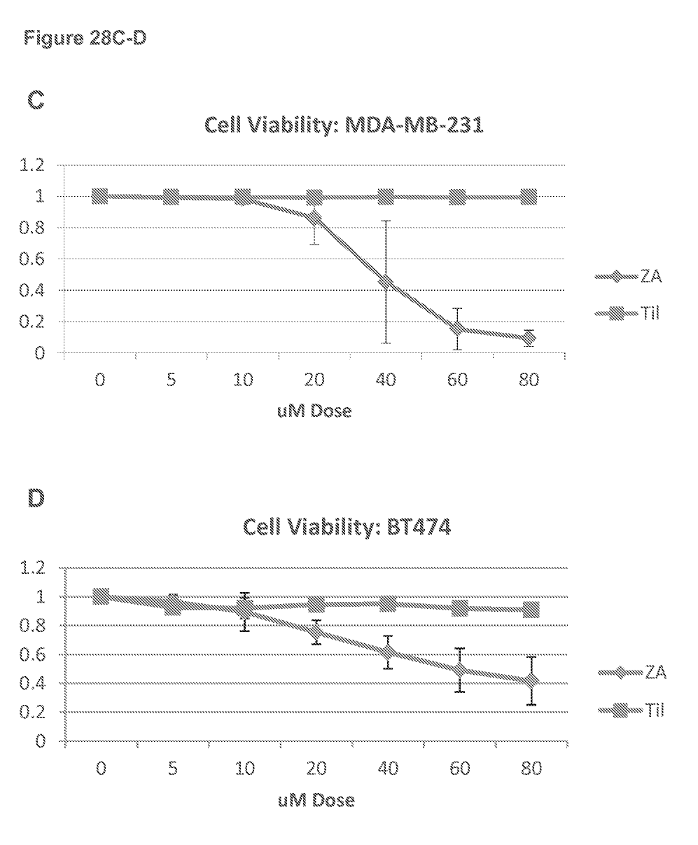

We also performed a cell-free in vitro kinase assay to determine if bisphosphonates could inhibit Her2 activity. Briefly, Ert, ZA, Ris, MA, or Til (doses as shown in FIG. 28A) were incubated for 30 minutes with 50 ng recombinant Her2 or EGFR^{L858R} protein (GENWAY) at 4° C. and tyrosine kinase activity was measured. Following addition of assay buffer (SIGMA) which contained the poly(Glu, Tyr) (4:1) substrate and ATP, the kinase reaction was allowed to proceed for 30 minutes at 30° C. An equal volume of Kinase-Glo substrate (PROMEGA) was then added, and luminescence read manually (TD-20/20, TURNER DESIGN). Each of the bisphosphonates tested was able to inhibit Her2 tyrosine kinase activity in the cell-free assay.

We also tested the effect of zoledronic acid on breast cancer cells harboring either an EGFR mutation or a Her2 mutation. Briefly, MDA-MB-231 cells, which overexpress EGFR (see FIG. 28B), and BT474 cells, which overexpress Her2 (see FIG. 28B), were grown using standard protocols, and exposed to increasing concentrations of zoledronic acid. Thereafter, the cells were subject to the MTT assay to assess viability using a commercial kit. Zoledronic acid inhibited cell viability of the EGFR mutant breast cancer cell line (MDA-MB-231) and the HER2 mutant breast cancer cell line (BT474) in a concentration-dependent manner (FIGS. 28C and 28D, respectively), suggesting that the effect of bisphosphonates is not specific to type I EGFR, but also extends to other receptor tyrosine kinases (RTKs), such as type II EGFR (Her2).

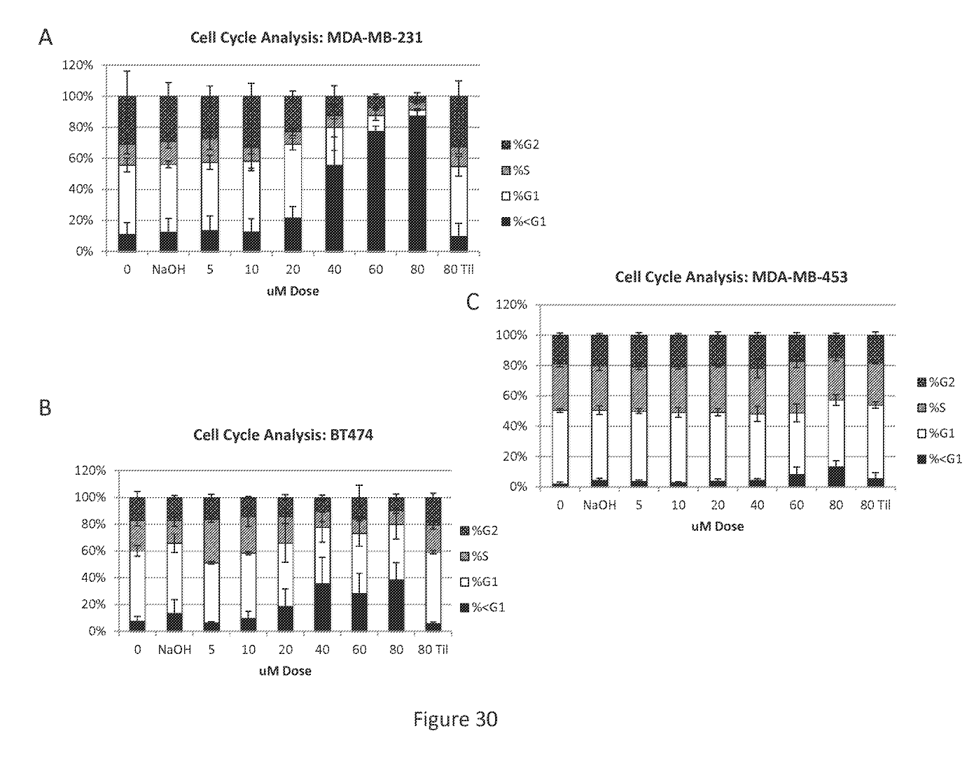

To examine the underlying cellular mechanism through which bisphosphonates decrease cell viability in breast cancer cell lines, we performed FACS analysis using EGFR-expressing (MDA-MB-231), HER2-expressing (BT474) and non-EGFR, non-HER2-expressing (MDA-MB-453) breast cancer cell lines grown in the presence of increase concentrations of Zoledronic Acid. Zoledronic acid increased the sub-G1 fractions in the MDA-MB-453 (FIG. 30A) and BT474 (FIG. 30B) cell lines, but not the MDA-MB0453 cell line (FIG. 30C), demonstrating a dramatic, concentration-dependent induction of apoptosis in EGFR- and HER2-dependant breast cancers. The effect of zoledronic acid on apoptosis was mirrored by a marked concentration-dependent (up to 20 μM ZA) inhibition of colony formation in MDA-MB-231 cells, which was not seen with tiludronate (20 μM) (data not shown).

SEQ ID NO: 1
(Human EGFR/ERBB1/HER1 Amino Acid Sequence)
MRPSGTAGAALLALLAALCPASRALEEKKVCQGTSNKLTQLGTFEDHFLS
LQRMFNNCEVVLGNLEITYVQRNYDLSFLKTIQEVAGYVLIALNTVERIP
LENLQIIRGNMYYENSYALAVLSNYDANKTGLKELPMRNLQEILHGAVRF
SNNPALCNVESIQWRDIVSSDFLSNMSMDFQNHLGSCQKCDPSCPNGSCW
GAGEENCQKLTKIICAQQCSGRCRGKSPSDCCHNQCAAGCTGPRESDCLV
CRKFRDEATCKDTCPPLMLYNPTTYQMDVNPEGKYSFGATCVKKCPRNYV
VTDHGSCVRACGADSYEMEEDGVRKCKKCEGPCRKVCNGIGIGEFKDSLS
INATNIKHFKNCTSISGDLHILPVAFRGDSFTHTPPLDPQELDILKTVKE
ITGFLLIQAWPENRTDLHAFENLEIIRGRTKQHGQFSLAVVSLNITSLGL
RSLKEISDGDVIISGNKNLCYANTINWKKLFGTSGQKTKIISNRGENSCK
ATGQVCHALCSPEGCWGPEPRDCVSCRNVSRGRECVDKCNLLEGEPREFV
ENSECIQCHPECLPQAMNITCTGRGPDNCIQCAHYIDGPHCVKTCPAGVM
GENNTLVWKYADAGHVCHLCHPNCTYGCTGPGLEGCPTNGPKIPSIATGM
VGALLLLLVVALGIGLFMRRRHIVRKRTLRRLLQERELVEPLTPSGEAPN
QALLRILKETEFKKIKVLGSGAFGTVYKGLWIPEGEKVKIPVAIKELREA
TSPKANKEILDEAYVMASVDNPHVCRLLGICLTSTVQLITQLMPFGCLLD
YVREHKDNIGSQYLLNWCVQIAKGMNYLEDRRLVHRDLAARNVLVKTPQH
VKITDFGLAKLLGAEEKEYHAEGGKVPIKWMALESILHRIYTHQSDVWSY
GVTVWELMTFGSKPYDGIPASEISSILEKGERLPQPPICTIDVYMIMVKC
WMIDADSRPKFRELIIEFSKMARDPQRYLVIQGDERMHLPSPTDSNFYRA
LMDEEDMDDVVDADEYLIPQQGFFSSPSTSRTPLLSSLSATSNNSTVACI
DRNGLQSCPIKEDSFLQRYSSDPTGALTEDSIDDTFLPVPEYINQSVPKR
PAGSVQNPVYHNQPLNPAPSRDPHYQDPHSTAVGNPEYLNTVQPTCVNST
FDSPAHWAQKGSHQISLDNPDYQQDFFPKEAKPNGIFKGSTAENAEYLRV
APQSSEFIGA
SEQ ID NO: 2
(Human HER2 Amino Acid Sequence)
MELAALCRWGLLLALLPPGAASTQVCTGTDMKLRLPASPETHLDMLRHLY
QGCQVVQGNLELTYLPTNASLSFLQDIQEVQGYVLIAHNQVRQVPLQRLR
IVRGTQLFEDNYALAVLDNGDPLNNTTPVTGASPGGLRELQLRSLTEILK
GGVLIQRNPQLCYQDTILWKDIFHKNNQLALTLIDTNRSRACHPCSPMCK
GSRCWGESSEDCQSLTRTVCAGGCARCKGPLPTDCCHEQCAAGCTGPKHS
DCLACLHFNHSGICELHCPALVTYNTDTFESMPNPEGRYTFGASCVTACP
YNYLSTDVGSCTLVCPLHNQEVTAEDGTQRCEKCSKPCARVCYGLGMEHL
REVRAVTSANIQEFAGCKKIFGSLAFLPESFDGDPASNTAPLQPEQLQVF
ETLEEITGYLYISAWPDSLPDLSVFQNLQVIRGRILHNGAYSLTLQGLGI
SWLGLRSLRELGSGLALIHHNTHLCFVHTVPWDQLFRNPHQALLHTANRP
EDECVGEGLACHQLCARGHCWGPGPTQCVNCSQFLRGQECVEECRVLQGL
PREYVNARHCLPCHPECQPQNGSVTCFGPEADQCVACAHYKDPPFCVARC
PSGVKPDLSYMPIWKFPDEEGACQPCPINCTHSCVDLDDKGCPAEQRASP
LTSIISAVVGILLVVVLGVVFGILIKRRQQKIRKYTMRRLLQETELVEPL
TPSGAMPNQAQMRILKETELRKVKVLGSGAFGTVYKGIWIPDGENVKIPV
AIKVLRENTSPKANKEILDEAYVMAGVGSPYVSRLLGICLTSTVQLVTQL
MPYGCLLDHVRENRGRLGSQDLLNWCMQIAKGMSYLEDVRLVHRDLAARN
VLVKSPNHVKITDFGLARLLDIDETEYHADGGKVPIKWMALESILRRRFT
HQSDVWSYGVTVWELMTFGAKPYDGIPAREIPDLLEKGERLPQPPICTID
VYMIMVKCWMIDSECRPRFRELVSEFSRMARDPQRFVVIQNEDLGPASPL
DSTFYRSLLEDDDMGDLVDAEEYLVPQQGFFCPDPAPGAGGMVHHRHRSS
STRSGGGDLTLGLEPSEEEAPRSPLAPSEGAGSDVFDGDLGMGAAKGLQS
LPTHDPSPLQRYSEDPTVPLPSETDGYVAPLTCSPQPEYVNQPDVRPQPP
SPREGPLPAARPAGATLERPKTLSPGKNGVVKDVFAFGGAVENPEYLTPQ
GGAAPQPHPPPAFSPAFDNLYYWDQDPPERGAPPSTFKGTPTAENPEYLG
LDVPV

1. A method of treating cancer, comprising
identifying a cancer subject who has a mutation in an epidermal growth factor receptor (EGFR) family member, and
administering to the subject an EGFR family member antagonist and a bisphosphonate that can bind to the EGFR family member.

2. The method of claim 1, wherein the EGFR family member has a mutation in its catalytic domain.

3. The method of claim 1, wherein the EGFR family member is an EGFR.

4. The method of claim 3, wherein the EGFR is overexpressed; or has one or more of the following mutations: L858R, an exon 20 insertion, a deletion in exon 19, and T790M.

5. (canceled)

6. The method of claim 1, wherein the EGFR family member is a human epidermal growth factor receptor 2 (HER2).

7. The method of claim 6, wherein the HER2 is overexpressed; has one or more of the following mutations: T798M, T798I, G309A, G309E, S310F, R678Q, L755S, L755W, I767M, D769H, D769Y, V777L, P780ins, V835F, V842I, R896C, and G1201V; or has an in-frame deletion.

8. The method of claim 1, wherein the bisphosphonate comprises one or more nitrogen atoms.

9. The method of claim 8, wherein the bisphosphonate comprises an imidazole ring.

10. The method of claim 8, wherein the bisphosphonate is zoledronic acid, minodronic acid or a salt thereof.

11. (canceled)

12. The method of claim 8, wherein the bisphosphonate is selected from the group consisting of alendronate, ibandronate, risedronate, and incadronate; and acids of the foregoing.

13. The method of claim 1, wherein the EGFR family member antagonist binds to the catalytic domain of the EGFR family member.

14. The method of claim 13, wherein the EGFR family member antagonist is erlotinib, gefitinib, lapatinib, neratinib or afatinib.

15. The method of claim 1, wherein the EGFR family member antagonist binds to an extracellular domain of the EGFR family member.

16. The method of claim 15, wherein the EGFR family member antagonist is cetuximab, panitumumab, matuzumab, nimotuzumab, trastuzumab, pertuzumab, or nelipepimut-S.

17. The method of claim 1, wherein the cancer is lung cancer, colorectal cancer, breast cancer, ovarian cancer, stomach cancer, or uterine cancer.

18-20. (canceled)

21. The method of claim 1, wherein the administering step is performed before the cancer metastasizes in the subject.

22. (canceled)

23. The method of claim 1, wherein the subject is refractory to treatment with the EGFR family member antagonist.

24-25. (canceled)

26. A method of enhancing the efficacy of an EGFR family member antagonist in a subject, comprising administering to the subject the EGFR family member antagonist and a bisphosphonate that can bind to the EGFR family member.

27-28. (canceled)

29. A method of treating cancer in a subject, wherein the cancer is characterized by a T790M mutation in EGFR, or a T798M or T798I mutation in HER2, comprising administering to the subject a bisphosphonate that binds to an EGFR family member.

30-34. (canceled)

35. A pharmaceutical composition comprising an EGFR family member antagonist and a bisphosphonate.

36-55. (canceled)


SEQUENCE LISTING (2 sequences)

SEQ ID NO: 1 - protein, 1210 aa, Homo sapiens [sequence omitted]

SEQ ID NO: 2 - protein, 1255 aa, Homo Sapiens [sequence omitted]